(19) United States
(12) Patent Application Publication
(10) Pub. No.: US 2016/0377599 A1
(43) Pub. Date: Dec. 29, 2016

(54) LIVER INFECTION

(71) Applicant: CN BIO INNOVATIONS LIMITED, Begbroke Oxfordshire (GB)

(72) Inventors: David Hughes, Hatfield, Hertfordshire (GB); Cliff Rowe, Enstone, Chipping Norton Oxfordshire (GB); Emma Large, Oxford, Oxfordshire (GB); Mark Thursz, Berkhamsted, Hertfordshire (GB); Marcus Dorner, London (GB)

(21) Appl. No.: 14/901,761

(22) PCT Filed: Jul. 11, 2014
(86) PCT No.: PCT/GB2014/052131

§ 371 Date: Dec. 29, 2015

(30) Foreign Application Priority Data
Jul. 12, 2013 (GB) 1312604.0
Oct. 17, 2013 (GB) 1318365.2
May 9, 2014 (GB) 1408266.3

(51) Int. Cl. G01N 33/50; C12N 7/00

(52) U.S. Cl. CPC G01N 33/5067; G01N 33/5082; C12N 7/00; G01N 2800/26; G01N 2333/02; C12N 2730/10121

(57) ABSTRACT

The invention relates to a method for studying an infection process in liver tissue in vitro, the method comprising: seeding hepatocyte cells onto a scaffold in a bioreactor in order to form a liver tissue model;delivering an infectious agent to the liver tissue model, or providing the liver tissue model pre-infected with an infectious agent; and monitoring the infection process; and a method for producing an infectious agent.

This invention relates to a method for studying infection of liver tissue in vitro, a screening method for potential therapeutic or preventative drug candidates, a liver infection model, a method for producing an infectious agent and progeny thereof.

Infectious liver disease is a major concern worldwide. For example, hepatitis B virus (HBV) is estimated to chronically infect 350 million individuals, which is a major global healthcare challenge. Study of viral infection is essential to understand the pathogenic mechanisms and to identify and develop novel therapeutics to combat the infection. However, the study of virus infection of the liver is difficult due to the limited availability of suitable viral infection and replication models. For example, the in vitro study of hepatitis B virus is often limited to infection of hepatocellular carcinoma cell lines, such as HepaRG or Huh 7, where viral particle uptake is poor and both infection and propagation of hepatotropic viruses require specific host factors that are mainly expressed in highly differentiated cells of the host species or a very limited number of other species. These highly differentiated cells rapidly lose aspects of their differentiation in cell culture resulting in problems ranging from low viral titres to a failure to infect the cells in cell culture. Study of hepatotropic viruses therefore has relied on highly modified cellular or viral systems: for example the use of induced pluripotent stems cells for the study of hepatitis C (Schwartz et al, PNAS, January 2012 http://www.pnas.org/content/early/2012/01/27/1121400109). Viral uptake inefficiency is often circumvented by plasmid DNA delivery into the cells. Such a method does not allow the study of viral entry. Furthermore, the cancer cell lines are not an ideal study model as they have abnormal differentiation, deregulated gene expression, aberrant cell signalling and endocytic functions. However, studies with the natural host of hepatitis B virus, namely primary human hepatocytes, are difficult, because they can rapidly de-differentiate and lose their capacity for hepatitis B virus infection and replication after a brief period in cell culture. In the case of hepatitis C virus (HCV), in vitro study can only be carried out on a limited subset of virus strains, limited to a recombinant 2a genome, JFH1. Furthermore, mutations that had been made in this genome, allowing replication in cell culture, have been demonstrated to interfere with the production of infectious virus (Pietschmann et al: PLoS Pathogens 5(6) 2009). Primary HCV isolates do not replicate well in cell culture and a major challenge in HCV biology remains to propagate additional genotypes or wild-type virus in vitro (Steinmann and Pietschmann: Current Topics in Microbiology and Immunology Volume 369, 2013, pp 17-48) Ploss et al (2010, PNAS, Vol 107(7) pp. 3141-3145) discusses strategies for hepatitis C infection models, which include extracellular matrix manipulations, defined culture media, fluid flow using bioreactors, or alteration of cell-cell interactions by forming 3D spheroidal aggregates or co-cultivation with nonparenchymal cell types. Ploss et al., further describes that although some of these models provide necessary extracellular matrix cues, they lack crucial heterotypic cell-cell interactions or control over tissue architecture, known to affect liver-specific functions. Importantly, the model investigated by Ploss et al., was poor, with the cells unable to provide efficient uptake, generating low titres, and spread of the virus infection to new cells was limited. It was postulated that correct polarisation and maintenance of differentiation of the hepatocytes may be important factors. The model used in Ploss et al. was also non-representative of a human system, using an heterologous combination of human an murine cells. Cell-cell interactions and potentially transmission have been implicated in hepatotropic virus reproduction and spread (summarised in Steinmann), demonstrating a need for an homologous system for these studies. Furthermore Wu et al., (2008. PLoS Pathogens Vol 8(4), pp. 1-14) discusses the importance of differentiation, where productive hepatitis C virus infection of stem cell-derived hepatocytes revealed a critical transition to viral permissiveness during differentiation.

An aim of the present invention is to provide improved methods for modelling liver infection process in vitro.

According to a first aspect of the invention, there is provided a method for studying an infection process in liver tissue in vitro, the method comprising:
seeding hepatocyte cells onto a scaffold in a bioreactor in order to form a liver tissue model;
delivering an infectious agent to the liver tissue model, or providing the liver tissue model pre-infected with an infectious agent; and
monitoring the infection process.

Delivering the infectious agent to the liver tissue may comprise adding the infectious agent to the liver tissue model after seeding of the hepatocyte cells. Delivering the infectious agent to the liver tissue may comprise adding the infectious agent to hepatocyte cells prior to seeding. Delivering the infectious agent to the liver tissue may comprise adding the infectious agent to non-parenchymal cells and subsequently seeding the non-parenchymal cells onto the scaffold. Providing the liver tissue model pre-infected with an infectious agent may comprise adding the infectious agent to hepatocyte and/or non-parenchymal cells prior to seeding them onto the scaffold.

The method may be for studying a viral infection and/or viral replication in liver tissue in vitro. Alternatively, the method may be for studying a malarial infection in liver tissue in vitro.

Advantageously, the method of the invention allows hepatocyte cells to maintain their differentiation over a long study period, such as over 14 days and even up to and beyond 28 days. Additionally, the method of the invention allows the cells to generate a more polarised phenotype, which has been demonstrated to have implications in infection, such as viral infection. The method of the invention provides an ideal environment, which mimics the natural host environment and leads to an in vitro model for studying the infection process.

The method may further comprise the delivery of an active agent to the liver tissue model. Combinations of active agents may be used concurrently or sequentially. The active agent may comprise a drug candidate, a marker, or a cell signalling molecule, such as a cytokine. The candidate drug may comprise an antibody, or fragment or variant thereof; a small molecule; a peptide; or nucleic acid, such as siRNA. The candidate drug may comprise an oligomer. The candidate drug may comprise a nucleotide analogue. The candidate drug may comprise an antiviral. The candidate drug may comprise an antibiotic.

A small molecule may comprise a chemical compound. A small molecule may not be a biological molecule. The small molecule may not be any one of a polymeric nucleic acid, a protein, or an antibody. A small molecule may comprise a low molecular weight <900 Daltons, organic compound that may serve as an enzyme substrate or regulator of biological processes, with a size on the order of 10⁻⁹ m. Biopolymers such as nucleic acids, proteins, and polysaccharides (such as starch or cellulose) may not be considered small molecules. The constituent monomers of biopolymers, such as ribo- or deoxyribonucleotides, amino acids, and monosaccharides may be considered to be small molecules. Small oligomers may be considered small molecules, such as dinucleotides, peptides such as the antioxidant glutathione, and disaccharides such as sucrose.

Delivering an active agent to the liver tissue model advantageously allows the screening and study of potential therapies for liver viral infection in vitro.

The active agent may be delivered to the liver tissue model before, concurrently, simultaneously, or after delivery of the virus, or nucleic acid encoding the virus.

The hepatocyte cells may be human or non-human. The hepatocyte cells may be human. The hepatocyte cells may comprise primary hepatocytes. The hepatocyte cells may comprise non-primary hepatocytes, such as stem cell derived hepatocytes or progenitor cell derived hepatocytes. The hepatocyte cells may be freshly-isolated from a donor, or from a cryopreserved source. The hepatocyte cells may be from a cryopreserved source. The hepatocyte cells may be from the same donor or combined from multiple donors. The hepatocyte cells may comprise or consist of female derived hepatocytes. The hepatocyte cells may comprise or consist of male derived hepatocytes. The hepatocyte cells may comprise or consist of fetal derived hepatocytes. The hepatocyte cells may comprise or consist of adult derived hepatocytes. The hepatocyte cells may comprise or consist of juvenile derived hepatocytes. Alternatively, the hepatocyte cells may not comprise or consist of fetal derived hepatocytes.

The method may further comprise seeding additional cells with the hepatocytes. The additional cells may comprise non-parenchymal liver cells.

The non-parenchymal liver cells may be selected from any of the group comprising macrophages (Kupffer cells); stellate cells (Ito cells); cholangiocytes; sinusoidal endothelial cells; liver precursor cells; and other, minor, non-hepatocyte liver cells; or combinations thereof. The non-parenchymal liver cells may be human or non-human. The non-parenchymal liver cells may be human. The non-parenchymal liver cells may be freshly-isolated from a donor, or from a cryopreserved source. The non-parenchymal liver cells may be from the same donor or combined from multiple donors. The non-parenchymal liver cells may comprise or consist of female derived non-parenchymal liver cells. The non-parenchymal liver cells may comprise or consist of male derived non-parenchymal liver cells. The non-parenchymal liver cells may comprise or consist of fetal derived non-parenchymal liver cells. The non-parenchymal liver cells may comprise or consist of adult derived non-parenchymal liver cells. The non-parenchymal liver cells may comprise or consist of juvenile derived non-parenchymal liver cells. Alternatively, the non-parenchymal liver cells may not comprise or consist of fetal derived non-parenchymal liver cells.

The non-parenchymal cells may be seeded onto the scaffold before, simultaneously, concurrently or after the hepatocyte cells. The non-parenchymal cells may be mixed with the hepatocyte cells prior to seeding onto the scaffold. The non-parenchymal cells may be seeded onto the scaffold simultaneously or concurrently with the hepatocyte cells.

The ratio of hepatocytes and non-parenchymal cells may be adjustable as required. The ratio of hepatocytes and non-parenchymal cells may be adjusted to model the ratio typically found in liver tissue. The hepatocytes and non-parenchymal cells may be sorted by FACS sorting. The ratio of hepatocytes to non-parenchymal cells may reflect a healthy or a diseased liver. The ratio of hepatocytes to Kupffer cells may be about 10:1, for example in a model of a healthy liver. The ratio of hepatocytes to Kupffer cells may be about 3:1, for example in a model of an inflamed liver. The ratio of hepatocytes to non-parenchymal cells may be between about 2:1 and about 20:1, or between about 3:1 and about 15:1. The ratio of hepatocytes to non-parenchymal cells may be between about 3:1 and about 10:1. The ratio of hepatocytes to non-parenchymal cells may be about 3:2.

The hepatocyte and non-parenchymal cells may be of the same species. The hepatocyte and non-parenchymal cells may be of the same development or differentiation stage. The hepatocyte and non-parenchymal cells may not be a heterologous combination of different species. The hepatocyte cells and non-parenchymal cells may comprise substantially all human cells, or essentially all human cells. The non-parenchymal cells may comprise substantially all human cells, or essentially all human cells. The hepatocyte cells may comprise substantially all human cells, or essentially all human cells.

The cells seeded may be substantially non-aggragated singlular cells. The cells may be provided in the form of spheroids. The cells may not be provided with a period of aggregation prior to, or during, seeding.

The hepatocytes and/or additional cells may be seeded into the liver tissue model at a density of between about 0.2×10⁶ and about 2×10⁶ cells per scaffold. The hepatocytes and/or additional cells may be seeded into the liver tissue model at a density of between about 0.3×10⁶ and about 2×10⁶ cells per scaffold. The hepatocytes and/or additional cells may be seeded into the liver tissue model at a density of between about 0.4×10⁶ and about 2×10⁶ cells per scaffold. The hepatocytes and/or additional cells may be seeded into the liver tissue model at a density of between about 0.5×10⁶ and about 2×10⁶ cells per scaffold. The hepatocytes and/or additional cells may be seeded into the liver tissue model at a density of between about 0.6×10⁶ and about 1.5×10⁶ cells per scaffold. The hepatocytes and/or additional cells may be seeded into the liver tissue model at a density of between about 0.6×10⁶ and about 1.2×10⁶ cells per scaffold. The hepatocytes and/or additional cells may be seeded into the liver tissue model at a density of between about 0.4×10⁶ and about 0.8×10⁶ cells per scaffold. The hepatocytes and/or additional cells may be seeded into the liver tissue model at a density of between about 0.5×10⁶ and about 0.8×10⁶ cells per scaffold. The hepatocytes and/or additional cells may be seeded into the liver tissue model at a density of between about 0.5×10⁶ and about 0.7×10⁶ cells per scaffold. The hepatocytes and/or additional cells may be seeded into the liver tissue model at a density of about 0.6×10⁶ cells per scaffold. The hepatocytes and/or additional cells may be seeded into the liver tissue model at a density of between about 10,000 and about 5,000,000 cells. The hepatocytes and/or additional cells may be seeded into the liver tissue model at a density of between about 50,000 and about 2,000,000 cells. The method may further comprise the addition of additional hepatocyte cells to the liver tissue model. The additional hepatocyte cells may be added during the cell culture, or during a media changeover/replacement.

The non-parenchymal cells may be infected with an infectious agent prior to the seeding onto the scaffold. The non-parenchymal cells may be infected with a virus, or nucleic acid encoding a virus, prior to the seeding onto the scaffold.

The infectious agent may be delivered between about 1 second and about 27 days before the active agent. The infectious agent may be delivered between about 1 minute and about 20 days before the active agent. The infectious agent may be delivered at least 1 day before the active agent. The infectious agent may be delivered at least 1 hour, or at least 3 hours, or at least 8 hours, or at least 12 hours, before the active agent.

The infectious agent may be delivered simultaneously or concurrently with the active agent. The infectious agent may be delivered between about 1 second and about 27 days after the active agent. The infectious agent may be delivered between about 1 minute and about 20 days after the active agent. The infectious agent may be delivered at least 1 day after the active agent. The infectious agent may be delivered at least 1 hour, or at least 3 hours, or at least 8 hours, or at least 12 hours, after the active agent.

Where the cells (such as hepatic cells—hepatocyte cells and/or non-parenchymal cells) are subject to pre-infection before seeding, the infectious agent may be delivered to the cells up to 6 hours before seeding. The infectious agent may be delivered to the cells up to 3 hours before seeding. The infectious agent may be delivered to the cells up to 1 hour before seeding.

The method may further comprise the delivery of an additional infectious agent. The additional infectious agent may be a different species, organism or strain from the first infectious agent, or a mutant of the infectious agent.

The method may further comprise inducing a cellular modification of one or more cells in the liver tissue model. The inducing of the cellular modification may be before, concurrent, simultaneous or after the infectious agent delivery. The inducing of the cellular modification may be before, concurrent, simultaneous or after the active agent delivery. The cellular modification may be a change in physiology, or a genetic modification, modifying genetic expression or regulation.

The infection process may produce progeny of the infectious agent. The progeny may be harvested. The progeny may be re-circulated in order to be delivered back to the cells of the liver tissue model.

The progeny may be attenuated. The progeny may be unable to replicate, or may be able to replicate only once. The progeny may be attenuated such that they are less pathogenic than an equivalent wild-type infectious agent that has not been attenuated. The infectious agent may comprise a virus, or nucleic acid encoding a virus. The infectious agent may comprise a parasite. The parasite may comprise a malarial parasite. The infectious agent may be of the genus Plasmodium. The infectious agent may comprise a protist. The infectious agent may comprise a sporozoite, merozoite, gametocyte or zygote/ookinete. The infectious agent may be bacterial, fungal or protozoan. The infectious agent may comprise any organism, virus, nucleic acid, or prion that is capable of affecting cells in liver tissue, infecting cells in liver tissue, replicating in cells in liver tissue, and/or carrying out at least part of its lifecycle in a cell in liver tissue. The infectious agent may comprise a viral vector. The viral vector may comprise HBV vector, for example derived from plasmid transfections in HepG2 cells.

The infectious agent may be capable of infecting cells of the liver, such as hepatocytes. The infectious agent may comprise any liver trophic infectious agent.

The virus may comprise a liver-affecting virus from a viral family selected from any of the group comprising Hepadnaviridae, Picornaviridae, Flaviviridae, Deltaviridae, and Hepeviridae.

The virus may comprise a member of the Hepadnaviridae family. The virus may comprise a hepatitis virus. The hepatitis virus may be selected from any of the group comprising hepatitis A (picornavirus), B (hepadnavirus), C (hepacivirus), D (deltavirus), E (hepevirus), F (currently hypothetical) and GB virus C. The virus may comprise hepatitis B, D or C virus. The virus may comprise hepatitis B virus. The virus may comprise hepatitis D virus. Combinations or mixtures of different viruses may be delivered to the liver tissue model. For example, two or more viruses selected from hepatitis A, B, C, D, E, F or G may be delivered to the liver tissue model. The virus may comprise HBeAg-negative HBV and/or HBeAg-positive HBV. The virus may comprise HCV genotype 1, 2, 3, 4, 5, 6, 7, 8, 9, 10, or 11. The virus may comprise HCV selected from any of the group comprising 1a, 1b, 1c, 2a, 2b, 2c, 3a, 3b, 4a, 4b, 4c, 4d, 4e, 5a, 6a, 7a, 7b, 8a, 8b, 9a, 10a, and 11a, or combinations thereof. The virus may comprise HCV genotype 2a. The virus may comprise HCV genotype 1a or 1b. The virus may comprise the HCV 2JFH1 strain.

The virus may comprise a liver-associated virus. A liver-associated virus may be considered to be a virus that is capable of infecting the liver or causing an effect in liver tissue, but the liver tissue may not be its primary tropism. The liver-associated virus may be selected from any of the group comprising cytomegalovirus, adenovirus, coxsackie virus, Echovirus, Rubella virus, Yellow Fever virus, Arenavirus, Marburg virus, Ebola virus, Rift Valley Fever virus, and Crimean Congo Hemorrhagic Fever-associated virus.

The infectious agent may comprise a drug resistant infectious agent. The infectious agent may comprise a drug resistant virus. A drug resistant virus may comprise antiviral resistant virus, such as DAA (directly acting antiviral) resistance. The infectious agent may comprise lamivudine, telaprevir, or boceprevir resistant virus. The infectious agent may comprise NS5A, NS5B, or NS3 HCV strains.

The infectious agent may comprise a modification wherein reporter/marker proteins are integrated in the infectious agent in addition to, or by substitution of, and infectious agent protein. The infectious agent may comprise a modification wherein reporter/marker proteins are encoded within the infectious agent nucleic acid in addition to, or by substitution of, infectious agent genetic code. The virus may comprise a modification wherein reporter/marker proteins are integrated in the virus particle in addition to, or by substitution of, viral protein. The virus may comprise a modification wherein reporter/marker proteins are encoded within the viral nucleic acid in addition to, or by substitution of, viral genetic code. The reporter/marker protein may comprise luciferase.

The infectious agent may be sourced from human blood product. The blood product may be from patients infected with the infectious agent. The blood product may be from a transgenic animal infected with the infectious agent. The virus may be sourced from transgenic animal models that produce human-infecting infectious agent.

The infectious agent may be concentrated prior to delivery. The infectious agent may be concentrated by precipitation. The infectious agent may be fully, substantially or partially purified prior to delivery.

Viral nucleic acid, such as DNA, may be transfected into the cells. The transfection may be prior to seeding the cells, or whilst the cells are on the scaffold. Where transfection occurs whilst the cells are on the scaffold, the flow of media may be turned off for an appropriate period to allow the transfection to take place. Viral nucleic acid may be in the form of a plasmid containing the viral genome, or parts thereof. Where the virus is delivered or infected by transfection of nucleic acid, the nucleic acid may be transcribed and translated to viral particles, or parts thereof.

The media may be changed/refreshed during the cell culture. The media may be changed prior to cells degrading to a non-differentiation state. The media may be changed between about 1 hours and about 36 hours after the cell seeding, or after about the first 24 hours. The media may be changed periodically, such as about every 24 hours, or about every 48 or about every 72 hours. The liver tissue model may be cultured for more than about 8 days. The liver tissue model may be cultured for more than about 14 days. The liver tissue model may be cultured for more than about 20 days or about 28 days. The liver tissue model may be cultured for more than about 40 days. The liver tissue model may be cultured for more than about 90 days. The period of culture may be between about 7 and about 14 days. The period of culture may be between about 7 and about 20 days. The period of culture may be between about 3 and about 14 days. The period of culture may be between about 1 and about 90 days. The period of culture may be for any period where the cell culture remains stable, such as where the hepatocyte cells remain differentiated.

Further additions of infectious agent may be delivered to the liver tissue model. Further additions of infectious agent may be provided during a media change. A single addition or multiple additions of infectious agent may be delivered at one or more media changes. Further additions of a different infectious agent may be delivered to the liver tissue model. For example the addition of hepatitis D virus to cells infected with, or exposed to, hepatitis B virus.

The virus may be delivered in an amount of at least 1 pfu. The virus may be delivered in an amount of at least 10 pfu/ml. The virus may be delivered in an amount of between about 10 pfu/ml and about 1000 pfu/ml. The virus may be delivered in an amount of between about 10 pfu/ml and about 100 pfu/ml. The virus may be delivered in an amount of between about 1 pfu/ml and an amount which will infect the cell via physiologically normal mechanisms. The virus may be delivered in an amount of less than 100,000 pfu/ml. The virus may be delivered in an amount of less than 10,000 pfu/ml. The virus may be delivered in an amount of less than 1000 pfu/ml. The virus may be delivered in an amount of less than 100 pfu/ml. The virus may be delivered in an amount of less than 10 pfu/ml. The bacteria may be delivered in an amount of at least 1 cfu. The bacteria may be delivered in an amount of at least 10 cfu/ml. The bacteria may be delivered in an amount of between about 10 cfu/ml and about 1000 cfu/ml. The bacteria may be delivered in an amount of between about 10 cfu/ml and about 100 cfu/ml. The bacteria may be delivered in an amount of between about 1 cfu/ml and an amount which will infect the cell via physiologically normal mechanisms.

The infectious agent may be delivered in an amount of at least 1 individual cell, organism, particle or molecule. The infectious agent may be delivered in an amount of at least 10 cells, organisms, particles or molecules. The infectious agent may be delivered in an amount of between about 10 and 1000 cells, organisms, particles or molecules. The infectious agent may be delivered in an amount of between about 10 and about 100 cells, organisms, particles or molecules. The infectious agent may be delivered in an amount of between about 1 cell, organism, particle or molecule and an amount which will infect the cell via physiologically normal mechanisms.

The infectious agent may be delivered in a quantity of at least about 0.01 m.o.i (multiplicity of infection). The infectious agent may be delivered in a quantity of at least about 0.05 m.o.i. The infectious agent may be delivered in a quantity of at least about 0.075 m.o.i. The infectious agent may be delivered in a quantity of at least about 0.1 m.o.i. The infectious agent may be delivered in a quantity of at least about 0.5 m.o.i. The infectious agent may be delivered in a quantity of at least about 0.75 m.o.i.

The infectious agent may be delivered in a quantity of at least about 0.2 m.o.i. The infectious agent may be delivered in a quantity of at least about 0.075 m.o.i. The infectious agent may be delivered in a quantity of about 0.75 m.o.i. The infectious agent may be delivered in a quantity of between about 0.01 m.o.i. and about 100 m.o.i. The infectious agent may be delivered in a quantity of between about 0.01 m.o.i. and about 10 m.o.i. The infectious agent may be delivered in a quantity of between about 0.01 m.o.i. and about 5 m.o.i. The infectious agent may be delivered in a quantity of between about 0.01 m.o.i. and about 2 m.o.i. The infectious agent may be delivered in a quantity of between about 0.01 m.o.i. and about 1 m.o.i. The infectious agent may be delivered in a quantity of between about 0.05 m.o.i. and about 10 m.o.i. The infectious agent may be delivered in a quantity of between about 0.05 m.o.i. and about 2 m.o.i. The infectious agent may be delivered in a quantity of less than 1000 m.o.i. The infectious agent may be delivered in a quantity of less than 100 m.o.i. The infectious agent may be delivered in a quantity of less than 10 m.o.i. The infectious agent may be delivered in a quantity of less than 1 m.o.i. The infectious agent may be delivered in a quantity of less than 0.5 m.o.i. The infectious agent may be delivered in a quantity of less than 0.1 m.o.i. The infectious agent may be delivered in a quantity of less than 0.05 m.o.i. The infectious agent may be delivered in a quantity of between about 1×10⁻⁹ per ml to about 1×10⁷ per ml. The infectious agent may be delivered in a quantity of between about 1×10⁻⁸ per ml to about 1×10⁵ per ml.

The infectious agent may be delivered in a quantity of at least about 1e3 per ml. The infectious agent may be delivered in a quantity of at least about 1e4 per ml. The infectious agent may be delivered in a quantity of at least about 1e5 per ml. The infectious agent may be delivered in a quantity of at least about 1e6 per ml. The infectious agent may be delivered in a quantity of at least about 1e7 per ml. The infectious agent may be delivered in a quantity of about 1e4.4 per ml. The infectious agent may be delivered in a quantity of about 1e3 per ml and about 1e7 per ml. The infectious agent may be delivered in a quantity of less than about 1e10 per ml. The infectious agent may be delivered in a quantity of less than about 1e9 per ml. The infectious agent may be delivered in a quantity of less than about 1e8 per ml. The infectious agent may be delivered in a quantity of less than about 1e7 per ml. The infectious agent may be delivered in a quantity of less than about 1e6 per ml. The infectious agent may be delivered in a quantity of less than about 1e5 per ml. The infectious agent may be delivered in a quantity of less than about 1e4 per ml. The infectious agent may be delivered in a quantity of less than about 1e3 per ml. The infectious agent may be delivered in a quantity of less than about 1e2 per ml.

The monitoring of the infection process may be over a period of about 1 hours to about 27 days. The monitoring of the infection process may comprise determining any of the group comprising attachment/adsorption; membrane fusion; endocytosis; penetration; unpackaging; nucleus localisation; replication, such as viral nucleic acid replication and/or viral protein expression; infectious agent growth; cyst formation; infectious agent adaption; infection agent morphosis; suppression of cellular defence; shedding; budding; apoptosis; exocytosis; viral latency; cell surface modifications/presentation; cellular response; cell signalling; cellular regulation; antiviral response; and cell senescence or cell death; or combinations thereof.

The infectious agent replication may be determined by quantification of infectious agent nucleic acid in the culture media or in the cells. The nucleic acid may be DNA or

RNA. The nucleic acid may be cccDNA. cccDNA generation may be monitored and/or quantified. cccDNA may be sequenced. cccDNA may be monitored during a study of the effects of an active agent delivered to the liver tissue model. Viral replication may be monitored by a strand-specific RT-PCR assay. Quantification of infectious agent nucleic acid in the culture media or in the cells may be by quantitative real-time PCR.

Monitoring the infection process may comprise analysis of the cells and/or cell culture media. Analysis of the cells and/or culture media may comprise determining the presence or quantity of any of the group comprising a metabolite; a cellular marker; a viral infection marker; virus particles; infectious agents; infectious agent nucleic acid; and cell signalling molecules; or combinations thereof. Supernatant and/or cells may be sampled for monitoring the infection process.

Analysis of the cells and/or cell culture media may comprise the use of HPLC, ELISA, PCR, mass-spectrometry, an enzyme assay, PAGE, western blot, chromatography, a molecular probe, such as a peptide, an oligonucleotide, or antibody probe.

Analysis of the cells and/or cell culture media may comprise determining the presence or quantity or hepatitis surface antigen. Determining the presence or quantity of hepatitis surface antigen may be by use of an Enzyme Linked Immunosorbent Assay (ELISA).

Monitoring the infection process may comprise analysis of one or more cells in the liver tissue model, such as the hepatocyte cells and/or additional cells of the liver tissue model, such as non-parenchymal cells. Analysis of the cells in the liver tissue model may comprise analysis within the bioreactor, and/or analysis of cells removed from the bioreactor. Analysis of the cells in the liver tissue model may comprise determination of any of the group comprising cell phenotype variation; genetic regulation; genetic modifications; cell surface modifications; localisation; life cycle stages; cell viability; attachment/adsorption; membrane fusion; endocytosis of infectious agent; penetration; virus particle unpackaging; nucleus localisation; infectious agent replication; viral nucleic acid replication; viral protein expression; infectious agent-mediated suppression of cellular defence; shedding; budding; cell apoptosis; exocytosis; viral latency; cell surface modifications/presentation; cellular response; cell signalling; cellular regulation; antiviral response; and cell senescence or death; or combinations thereof.

Monitoring the infection process may comprise detection and/or quantification of replication intermediates of the infectious agent. Replication intermediates may comprise pgRNA and/or nucleic acid encoding viral transcription factors. Replication intermediates may comprise non-packaged nucleic acid of a virus. Monitoring the infection process may be selected from any of the group comprising analysing supernatant for HBV nucleic acid, analysing supernatant for HBeAg, analysing supernatant for cytokine production, and analysing supernatant for albumin secretion, or combinations thereof.

Monitoring the infection process may comprise analysing cells for any of the group comprising HBV nucleic acid, such as HBV cccDNA, replication intermediates, such as nucleic acid encoding for viral transcription factors, HBV intermediates and/or pgRNA; or combinations thereof.

Monitoring the infection process may comprise analysis of genetic and/or epigenetic changes of cells in the liver tissue model. Genetic and/or epigenetic changes may be up-regulation or down-regulation of expression.

Monitoring the infection process may comprise the use of fluorescence excitation of molecules of interest. Readouts of injury or infection can be based on changes in fluorescence of the tissue as detected by a miniaturized fiber optic array which excites fluorescence via either single or multiphoton means. Many types of fluorescent readouts are possible. Changes in basic metabolic parameters of the liver tissue model may be measured by detecting a change in NAD(P)H levels, for example via intrinsic fluorescence. Cells may be pre-loaded with a dye which leaks in the case of membrane damage, resulting in a decrease of fluorescent intensity. Reporter genes may be transfected into the cells under the control of a stress-related promoter which is activated during tissue injury to produce a fluorescent product.

Monitoring the infection process may be at each media change. Monitoring the infection process may be continuous or intermittent. Monitoring the infection process may be at least every 12 hours. Monitoring the infection process may be at least every 24 hours. Monitoring the infection process may be at least every 48 hours.

The bioreactor may comprise a bioreactor well comprising the scaffold disposed therein. The bioreactor well may comprise, or may be arranged to receive, cell culture media. The bioreactor may comprise a fluid reservoir fluidly connected to the bioreactor well. The bioreactor well and fluid reservoir may be connected by fluidic channels allowing recirculation of cell culture media.

Two or more bioreactors may be connected, such as fluidly connected. The two or more bioreactors may connected by a channel, or two or more channels. The connection between two or more bioreactors may be controllable, to permit or prevent flow between the bioreactors. The connection between two or more bioreactors may controlled by a gate or valve. All bioreactors in an array may be connected, such as fluidly connected. All bioreactors in an array may be connected by one or more channels.

The two or more bioreactors may contain the same liver tissue model. Alternatively, at least one of the bioreactors may comprise a liver tissue model and a least one other bioreactor may comprise a different tissue, different cell type or different tissue model, for example, gut, kidney or other tissue.

The scaffold may be supported on a perfusible membrane. The perfusible membrane may be permeable to the infectious agent particles, or impermeable to the infectious agent. The perfusible membrane may comprise a filter of, for example but not limited to, nitrocellulose or nylon. The filter may comprise commercially available filters, for example, Whatman 903 (W-903), Ahlstrom grade 226 (A-226) or Munktell TFN (M-TFN). The perfusible membrane may comprise commercially available filters, for example, Whatman paper no. 1, nitrocellulose membrane no. 71002 (Amersham), or (iii) HYBOND-M nylon membrane (Amersham). The perfusible membrane may comprise functional and/or physical properties substantially similar to the commercially available perfusible membranes described herein. The cell culture media may be flowed or perfused through the scaffold. The cell culture media may be flowed or perfused through the perfusible membrane.

The bioreactor may comprise a mechanism for circulating cell culture media. The bioreactor may comprise a pump mechanism for circulating cell culture media. The pump mechanism may comprise a pneumatic or electromagnetically driven pump. The pneumatic pump may comprise a flexible membrane. Circulating the cell culture media may be by the action of a pneumatic pump flexing a flexible membrane, which acts on a fluidic channel.

The scaffold may comprise microchannels, such as a plurality of microchannels. The scaffold may provide a capillary structure having microchannels. The microchannels may be about 0.30 mm in diameter. The microchannels may be between about 0.075 mm and about 0.4 mm in diameter. The microchannels may be greater than 0.02 mm in diameter. The microchannels may have a depth of about 1 mm or less. The microchannels may have a depth of about 0.5 mm or less. The microchannels may have a depth of about 0.25 mm. The scaffold structure may be formed, for example, by etching, punching, drilling, lithography or laser cutting. The scaffold may comprise polystyrene. The scaffold may be coated to enhance cell adherence, and/or enhance maintenance of differentiation of the cells. The scaffold coating may comprise extracellular matrix. The scaffold coating may comprise type I collagen or RGD peptide.

The liver tissue model may be a 3-dimensional liver tissue model wherein cells are arranged in space along 3-dimensions. The 3-D structure may be provided by the scaffold comprising microchannels. The liver tissue models may not be a 2-dimension monolayer with cells arranged in space along a single plane.

Two or more bioreactors may be provided in an array. The array may comprise three or more, four or more, five or more, six or more, seven or more, or eight or more bioreactors. The array may comprise at least ten bioreactors. The array may comprise a plurality of bioreactors.

Two or more bioreactors in an array may share a pump for circulating cell culture media. Two or more bioreactors in an array may share a pump control mechanism.

The liver tissue model may comprise the apparatus and/or tissue models substantially or essentially as described in patent publication number WO2005123950. The liver tissue model may comprise the apparatus substantially or essentially as described in patent publication number WO9947922. The disclosures of patent publication numbers WO2005123950 and WO9947922 are herein incorporated by reference.

The liver tissue model may be capable of maintaining differentiation of the hepatocyte cells for at least 5 days, for at least 10 days, for at least 14 days, for at least 20 days, for at least 25 days, or for at least 27 days.

The hepatocytes cells may be maintained in the physiologically correct polarity relative to the microchannels of the scaffold. The hepatocyte cells may comprise a canalicular surface. The hepatocyte cells may maintain NTCP receptor on the cell surface. The NTCP receptor may be on a canalicular surface of the hepatocyte cell. The hepatocyte cells may maintain NTCP receptor on the surface, such as the canalicular surface, for at least 8 days, for at least 14 days, for at least 20 days, or for at least 28 days.

The hepatocytes cells may be maintained with the flow rate of the medium maintained such that an oxygen gradient approximating to physiological is produced. Oxygen may be provided by absorption of oxygen into the media. The bioreactor may be arranged on a perfusion culture plate, allowing oxygen to perfuse into the cell culture media.

The hepatocyte cells may be seeded with a seeding media or cell culture media. The hepatocyte cells may be seeded with a seeding media. The media for seeding the cells and/or maintaining the liver tissue model may comprise a suitable cell culture media.

The seeding media may comprise Williams E medium (such as from Life Technologies—A1217601). The seeding media may comprise Williams E medium and a cocktail of supplements, FBS and dexamethasone (for example as provided in a plating supplement pack from Life Technologies, Invitrogen—CM3000). The seeding media may comprise Dulbecco's Eagle Medium, hepatocyte Long Term Medium or other commercially available or internally made medium. The cell culture media may comprise serum albumin. The serum albumin may be added after virus delivery or after virus infection. Adding the serum albumin after the virus delivery or infection may advantageously reduce the potential for the virus binding to the serum albumin.

The cell culture media/maintenance media may comprise Williams E medium. The cell culture media/maintenance media may comprise Williams E medium and a cocktail of supplements and dexamethasone (for example as provided in a plating supplement pack from Life Technologies, Invitrogen—CM4000). The cell culture media may comprise Dulbecco's Eagle Medium, hepatocyte Long Term Medium or other commercially available or internally made medium. The cell culture media may comprise serum albumin. The serum albumin may be added after virus delivery or after virus infection. Adding the serum albumin after the virus delivery or infection may advantageously reduce the potential for the virus binding to the serum albumin.

The cell culture media may comprise Williams E medium. The cell culture media may comprise Dulbecco's Eagle Medium, hepatocyte Long Term Medium or other commercially available or internally made medium.

The delivery of the virus to the liver tissue model may be with an infection media. The infection media may comprise a media compatible with the virus and the cells of the liver tissue model. The infection media may comprise an appropriate cell culture media. The infection media may comprise Williams E medium or Dulbecco's Eagle Medium or other commercially available medium.

Patient serum (potentially containing the virus) may be diluted prior to introduction into the liver tissue model. Patient serum (potentially containing the virus) may be neat prior to introduction into the liver tissue model. The infection media may be used to dilute the patient serum (potentially containing the virus) prior to introduction into the liver tissue model. The dilution of patient serum may be at least 2 fold prior to introduction into the liver tissue model. The dilution of patient serum may be at least 5 fold prior to introduction into the liver tissue model. The dilution of patient serum may be at least 10 fold prior to introduction into the liver tissue model. The dilution of patient serum may be at least 20 fold prior to introduction into the liver tissue model. The dilution of patient serum may be at least 100 fold prior to introduction into the liver tissue model. The dilution of patient serum may be at least 200 fold prior to introduction into the liver tissue model. The dilution of patient serum may be at least 500 fold prior to introduction into the liver tissue model. The dilution of patient serum may be at least 1000 fold prior to introduction into the liver tissue model. The dilution of patient serum may be at least 10,000 fold prior to introduction into the liver tissue model. The dilution of patient serum may be between about 5 fold and about 10,000 fold prior to introduction into the liver tissue model. The dilution of patient serum may be between about 2 fold and about 1000 fold prior to introduction into the liver tissue model. The dilution of patient serum may be between about 100 fold and about 500 fold prior to introduction into the liver tissue model. The dilution of patient serum may be about 250 fold prior to introduction into the liver tissue model. In one embodiment, 204.1 of patient serum may be diluted in 50 ml of culture medium.

The method may further comprise the induction of an immune response in the liver tissue model. The immune response may be induced by LPS (lipopolysaccharide) stimulation of the cells, such as Kupffer cells. Other methods of inducing an immune response may be available.

The flow rate of the media through each bioreactor well may be between about 0.2 μl/s and about 4.4 μl/s. The flow rate of the media through each bioreactor well may be between about 1 μl/s. The flow rate of the media through each bioreactor well may be between about 0.2 μl/s and about 37 μl/s. The flow rate of the media through each microchannel in the scaffold may be between about 0.04 μl/minute and about 0.88 μl/minute.

During cell seeding, e.g. the stage at which the cells are introduced into the scaffold, the flow rate of the media through each bioreactor well may be between about 0.2 μl/s and about 4.4 μl/s. During cell seeding, the flow rate of the media through each bioreactor well may be between about 0.5 μl/s and about 3 μl/s. During cell seeding, the flow rate of the media through each bioreactor well may be between about 0.5 μl/s and about 2 μl/s. During cell seeding, the flow rate of the media through each bioreactor well may be between about 0.8 μl/s and about 2.5 μl/s. During cell seeding, the flow rate of the media through each bioreactor well may be between about 1 μl/s and about 2 μl/s. During cell seeding, the flow rate of the media through each bioreactor well may be about 1 μl/s. The seeding may comprise flowing the seeding media through the bioreactor(s) at between about 0.2 μl/s and about 4.4 μl/s for at least about 6 hours. The seeding may comprise flowing the seeding media through the bioreactor(s) at between about 0.2 μl/s and about 4.4 μl/s for between about 8 hours and about 16 hours. The seeding may comprise flowing the seeding media through the bioreactor(s) at between about 0.2 μl/s and about 4.4 μl/s for about 8 hours. The seeding may comprise flowing the seeding media through the bioreactor(s) at 1 μl/s for about 8 hours. About 0.6×10⁶ cells per scaffold may be seeded at a flow rate of about 1 μl/s. Between about 0.4×10⁶ and about 1×10⁶ cells per scaffold may be seeded at a flow rate of about 1 μl/s. Between about 0.4×10⁶ and about 0.8×10⁶ cells per scaffold may be seeded at a flow rate of about 1 μl/s. Between about 0.5×10⁶ and about 0.7×10⁶ cells per scaffold may be seeded at a flow rate of about 1 μl/s. About 0.6×10⁶ cells per scaffold may be seeded at a flow rate of between about 0.5 μl/s and about 2 μl/s. About 0.6×10⁶ cells per scaffold may be seeded at a flow rate of between about 0.8 μl/s and about 1.8 μl/s. About 0.6×10⁶ cells per scaffold may be seeded at a flow rate of between about 0.9 μl/s and about 1.2 μl/s. Between about 0.4×10⁶ and about 1×10⁶ cells per scaffold may be seeded at a flow rate of between about 0.5 μl/s and about 2 μl/s. Between about 0.5×10⁶ and about 0.7×10⁶ cells per scaffold may be seeded at a flow rate of between about 0.9 μl/s and about 1.2 μl/s.

During cell culture, the flow rate of the media through each bioreactor well may be between about 0.2 μl/s and about 4.4 μl/s. During cell culture, the flow rate of the media through each bioreactor well may be between about 0.5 μl/s and about 3 μl/s. During cell culture, the flow rate of the media through each bioreactor well may be between about 0.5 μl/s and about 2 μl/s. During cell culture, the flow rate of the media through each bioreactor well may be between about 0.8 μl/s and about 2.5 μl/s. During cell culture, the flow rate of the media through each bioreactor well may be between about 1 μl/s and about 2 μl/s. During cell culture, the flow rate of the media through each bioreactor well may be about 1 μl/s.

A cell density and flow rate may be used as appropriate to avoid significant down regulation of differentiation factors, such as down regulation of the gene expressing NTCP.

The use of the liver tissue model may comprise a cell seeding stage at time 0 hrs, followed by a period of cell culture and infectious agent delivery. The seeding stage may be transitioned to the culture stage by a change in media, such as a change from seeding media to culture media. The seeding stage may be transitioned to the culture stage by a change in flow of the media, such as flow rate.

The cell seeding stage may comprise a flowing the seeding media down through the scaffold from time 0 hrs (e.g. a flowing down towards the top of the scaffold structure in the bioreactor) to encourage cells to embed and attach to the scaffold. The flow direction may be reversed after a time period to pump the media up through the scaffold structure in the bioreactor. The time period to reverse the flow of media may be at between about 4 hrs and about 12 hours. The time period to reverse the flow of media may be at between about 6 hrs and about 12 hours. The time period to reverse the flow of media may be at between about 8 hrs and about 12 hours. The time period to reverse the flow of media may be at about 8 hrs.

The seeding media may be changed to culture media at between about 12 hrs and about 48 hrs. The seeding media may be changed to culture media at between about 16 hrs and about 48 hrs. The seeding media may be changed to culture media at between about 20 hrs and about 48 hrs. The seeding media may be changed to culture media at between about 24 hrs and about 48 hrs. The seeding media may be changed to culture media at between about 16 hrs and about 32 hrs. The seeding media may be changed to culture media at about 24 hrs.

The use of the liver tissue model may comprise a cell seeding stage at time 0, wherein the infectious agent has already been introduced to the cells. The use of the liver tissue model may comprise a cell seeding stage at time 0, and delivery of the infectious agent at time 0. The infectious agent delivery may be at between about 48 hours and about 96 hours after seeding. The infectious agent delivery may be at between about 60 hours and about 85 hours after seeding. The infectious agent delivery may be at between about 65 hours and about 80 hours after seeding. The infectious agent delivery may be at between about 70 hours and about 75 hours after seeding. The infectious agent delivery may be at about 72 hours after seeding.

The seeded cells may be washed after the delivery of the infectious agent. The wash may be at between about 1 hour and about 8 hours after delivery of the infectious agent. The wash may be at between about 2 hours and about 8 hours after delivery of the infectious agent. The wash may be at between about 3 hours and about 8 hours after delivery of the infectious agent. The wash may be at between about 3 hours and about 5 hours after delivery of the infectious agent. The wash may be at least about 3 hours after delivery of the infectious agent. The wash may be at about 3 hours after delivery of the infectious agent.

Monitoring, such as detection of viral replication, may take place at any time point.

The liver tissue model may be primed for use prior to seeding. The priming may comprise the flowing of seeding media through the scaffold at the relevant cell culture temperature (e.g. 37° C.) prior to seeding the cells. The priming may be provide at least 1 day, 2 days, or 3 days prior to seeding at 0 hrs.

The liver tissue model may produce or be capable of producing between 1000 and 100,000 TCID50/mL infectious agent per 48 hours. The liver tissue model may produce or be capable of producing between 1000 and 100,000 TCID50/mL infectious agent per 24 hours. The liver tissue model may produce or be capable of producing between 1000 and 100,000 TCID50/mL infectious agent per 12 hours. The liver tissue model may produce or be capable of producing between 1000 and 10,000 TCID50/mL infectious agent per 48 hours. The liver tissue model may produce or be capable of producing between 1000 and 10,000 TCID50/mL infectious agent per 24 hours. The liver tissue model may produce or be capable of producing between 1000 and 10,000 TCID50/mL infectious agent per 12 hours. The liver tissue model may produce or be capable of producing at least 1000 TCID50/mL infectious agent per 12 hours. The liver tissue model may produce or be capable of producing at least 10,000 TCID50/mL infectious agent per 12 hours. The liver tissue model may produce or be capable of producing at least 1000 TCID50/mL infectious agent per 24 hours. The liver tissue model may produce or be capable of producing at least 10,000 TCID50/mL infectious agent per 24 hours. The liver tissue model may produce or be capable of producing at least 50,000 TCID50/mL infectious agent per 24 hours. The liver tissue model may produce or be capable of producing at least 1000 TCID50/mL infectious agent per 48 hours. The liver tissue model may produce or be capable of producing at least 10,000 TCID50/mL infectious agent per 48 hours. The liver tissue model may produce or be capable of producing at least 50,000 TCID50/mL infectious agent per 48 hours. The liver tissue model may produce or be capable of producing at least 100,000 TCID50/mL infectious agent per 48 hours. The liver tissue model may produce or be capable of producing between 1000 and 100,000 TCID50/mL infectious agent per 24 hours or 48 hours, after at least 3 days post infection. The liver tissue model may produce or be capable of producing between 1000 and 100,000 TCID50/mL infectious agent per 24 hours or 48 hours, after at least 5 days post infection. The liver tissue model may produce or be capable of producing between 1000 and 100,000 TCID50/mL infectious agent per 24 hours or 48 hours, up to 7 days post infection. The liver tissue model may produce or be capable of producing between 1000 and 100,000 TCID50/mL infectious agent per 24 hours or 48 hours, up to 10 days post infection. The liver tissue model may produce or be capable of producing between 1000 and 100,000 TCID50/mL infectious agent per 24 hours or 48 hours, up to 14 days post infection. The liver tissue model may produce or be capable of producing between 1000 and 100,000 TCID50/mL infectious agent per 24 hours or 48 hours, up to 20 days post infection. The liver tissue model may produce or be capable of producing between 1000 and 100,000 TCID50/mL infectious agent per 24 hours or 48 hours, between about 3 days and about 7 days post infection.

The liver tissue model may produce or be capable of producing between 1000 and 100,000 TCID50/mL infectious agent per 24 hours or 48 hours, between about 3 days and about 14 days post infection.

The cell culture media, seeding media and infection media may not comprise DMSO, or

DMSO derivatives such as MSM or alternatives. DMSO alternative may include DTSSP (3,3′-dithiobis [sulfosuccinimidylpropionate]), DTBP (dimethyl 3,3′-dithiobispropionimidate), PEG, or organic solvents, such as methanol or ethanol. DMSO, or DMSO derivatives or alternatives may be not be provided in the liver tissue model of the invention. DMSO, or DMSO derivatives or alternatives may be not be provided in excess of 1% in the liver tissue model of the invention. DMSO, or DMSO derivatives or alternatives may be not be provided in excess of 0.5% in the liver tissue model of the invention. DMSO, or DMSO derivatives or alternatives may be not be provided in excess of 0.1% in the liver tissue model of the invention. DMSO, or DMSO derivatives or alternatives may be not be provided in excess of 0.01% in the liver tissue model of the invention. DMSO, or DMSO derivatives or alternatives may be not be provided in excess of 1% in the culture media, seeding media, or infection media. DMSO, or DMSO derivatives or alternatives may be not be provided in excess of 0.5% in the culture media, seeding media, or infection media. DMSO, or DMSO derivatives or alternatives may be not be provided in excess of 0.1% in the culture media, seeding media, or infection media. DMSO, or DMSO derivatives or alternatives may be not be provided in excess of 0.05% in the culture media, seeding media, or infection media. DMSO, or DMSO derivatives or alternatives may be not be provided in excess of 0.01% in the culture media, seeding media, or infection media. DMSO, or DMSO derivatives or alternatives may be not be provided in excess of 1% in the culture media. DMSO, or DMSO derivatives or alternatives may be not be provided in excess of 0.5% in the culture media. DMSO, or DMSO derivatives or alternatives may be not be provided in excess of 0.1% in the culture media. DMSO, or DMSO derivatives or alternatives may be not be provided in excess of 0.05% in the culture media. DMSO, or DMSO derivatives or alternatives may be not be provided in excess of 0.01% in the culture media. DMSO, or DMSO derivatives or alternatives may be not be provided in excess of 1% in the infection media. DMSO, or DMSO derivatives or alternatives may be not be provided in excess of 0.5% in the infection media. DMSO, or DMSO derivatives or alternatives may be not be provided in excess of 0.1% in the infection media. DMSO, or DMSO derivatives or alternatives may be not be provided in excess of 0.05% in the infection media. DMSO, or DMSO derivatives or alternatives may be not be provided in excess of 0.01% in the infection media.

According to another aspect of the invention, there is provided a screening method for identifying potential therapeutic or preventative drug candidates for the treatment or prevention of liver infection, the method comprising:
providing a liver tissue model, wherein the liver tissue model comprises hepatocytes adhered to a scaffold in a bioreactor;
delivering an infectious agent to the liver tissue model; or providing the liver tissue model pre-infected with an infectious agent;
delivering an active agent to the liver tissue model; and
monitoring the infection process.

Providing a liver tissue model may comprise seeding cells onto a scaffold in a bioreactor.

According to another aspect of the invention, there is provided an infection model of the liver for studying the infection process of an infectious agent capable of infecting the liver, the model comprising:
a liver tissue model, wherein the liver tissue model comprises hepatocytes adhered to a scaffold in a bioreactor; and
wherein the liver tissue model is infected with the infectious agent.

According to another aspect of the invention, there is provided a method of producing an infectious agent, the method comprising:
infecting a liver-tissue model with an infectious agent;
incubating the infected liver-tissue model to produce progeny of the infectious agent; and
harvesting the progeny.

The infectious agent may be a virus. The virus may be a hepatitis virus, or a modified hepatitis virus. The virus may be hepatitis A, B, C, D, E, F or G. The virus may be hepatitis B, D or C. The modified hepatitis virus may be attenuated. The modified hepatitis virus may be able to replicate in a hepatocyte cell to form virus progeny, but the virus progeny therefrom may be attenuated. The attenuation may be an inability to replicate, inability to enter the cell, or lack of or reduction in pathogenesis. The infectious agent may be any one of the infectious agents described herein.

According to another aspect of the invention, there is provided an infectious agent produced by the method according to the invention herein.

The infectious agent may be a virus.

According to another aspect of the invention, there is provided a method for identifying a novel biomarker for liver infection comprising the use of the method according to the invention herein, wherein the biomarker is determined by monitoring the infection process and identifying the presence, absence, increase or decrease of a molecule in response to the infection.

According to another aspect of the invention, there is provided a method for studying viral infection and/or replication in liver tissue in vitro, the method comprising:
seeding hepatocyte cells onto a scaffold in a bioreactor in order to form a liver tissue model;
delivering a virus, or nucleic acid encoding a virus, to the liver tissue model; or infecting the hepatocyte cells with a virus, or nucleic acid encoding a virus, prior to the seeding onto the scaffold; or infecting non-parenchymal hepatic cells with the virus, or nucleic acid encoding the virus, and subsequently seeding the non-parenchymal hepatic cells onto the scaffold; and
monitoring the viral infection process and/or life cycle.

A method for studying an infection process in liver tissue in vitro, the method comprising:
priming a scaffold in a bioreactor by flowing media through the scaffold at about 37° C. for at least about 12 hours;
   seeding hepatocyte cells onto a scaffold in a bioreactor in order to form a liver tissue model, wherein the hepatocyte cells are suspended in a seeding media, and the seeding media is flowed through the scaffold at about 1 μl/s at about 37° C. for about 24 hours;
changing the media to a cell culture media at about 24 hrs and flowing the cell culture media through the scaffold at a flow rate of 1 μl/s at about 37° C. in order to maintain a cell culture of the liver tissue model;
delivering an infectious agent to the liver tissue model;
washing the hepatocyte cells at 4 hours after delivering the infectious agent by performing a media change with cell culture media; and
monitoring the infection process.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

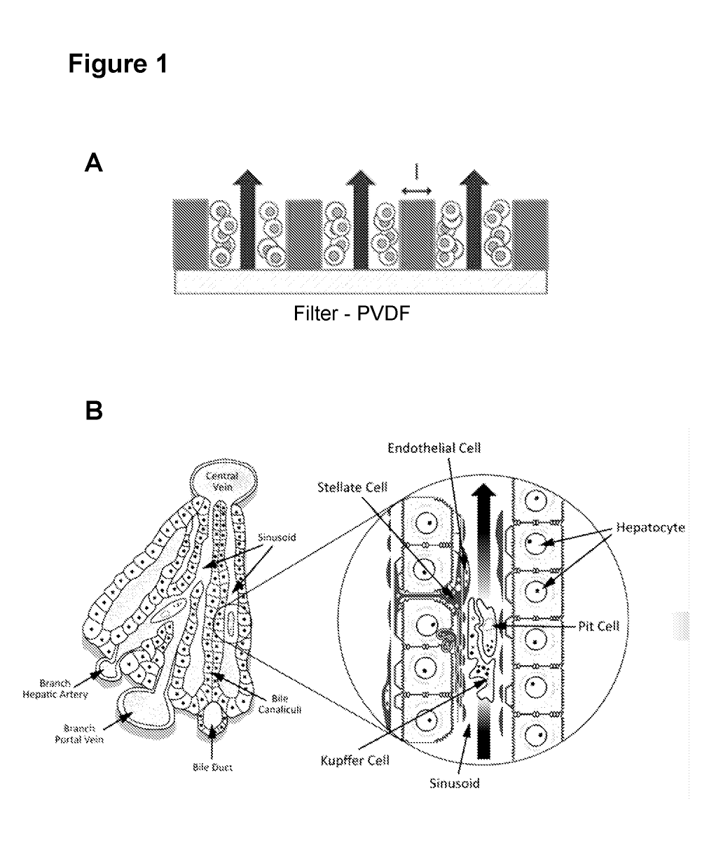

FIG. 1 Shows how the scaffold recreates the environment of the natural liver capillary bed in order to maintain function. FIG. 1A shows the scaffold, which has been engineered to recreate microtissues under flow which resemble the plate-like arrangement of hepatocytes and non-parenchymal cells aligned along the liver sinusoid. The scaffold is integrated with a perfusion plate enabling direct perfusion of the scaffold and in situ liver microtissues. FIG. 1B shows details of the natural liver capillary bed.

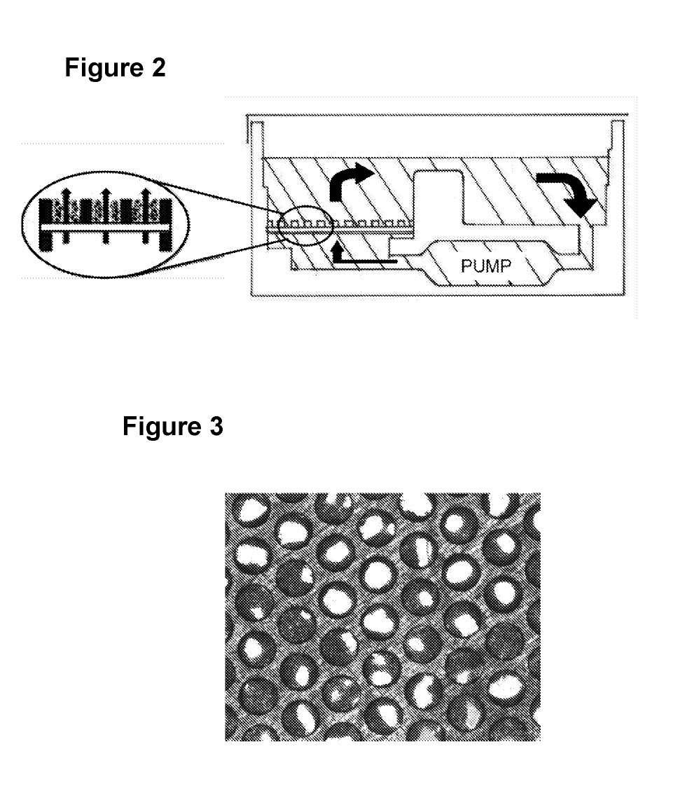

FIG. 2 shows the liver tissue model as it would be maintained in the bioreactor.

FIG. 3 demonstrates maintenance of the liver phenotype, where the natural sinusoidal architecture is replicated. A magnified view of the scaffold with human cells is shown.

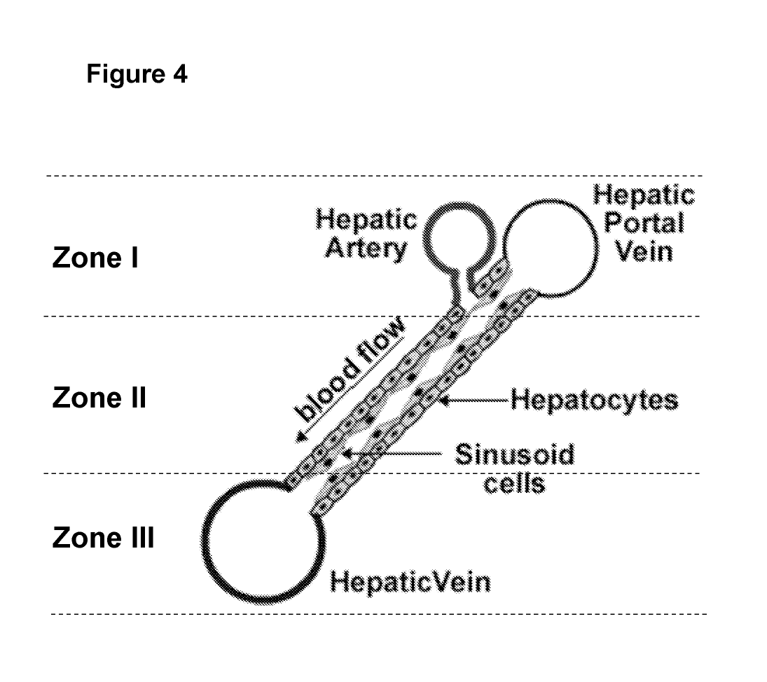

FIG. 4 is a schematic of natural liver tissue showing functional zonation and oxygen Tension. Zone I (periportal hepatocytes) is involved with gluconeogenesis, β-oxidation of fatty acids, and cholesterol synthesis. Zone II is the intermediate zone. Zone III (perivenous hepatocytes) is involved with glycolysis, lipogenesis, and cytochrome P-450. Oxygen tenation has been implicated in the replication of certain hepatotropic viruses [Vassilaki et al, Journal of Virology 87 (5) p 2935-2948. March 2013].

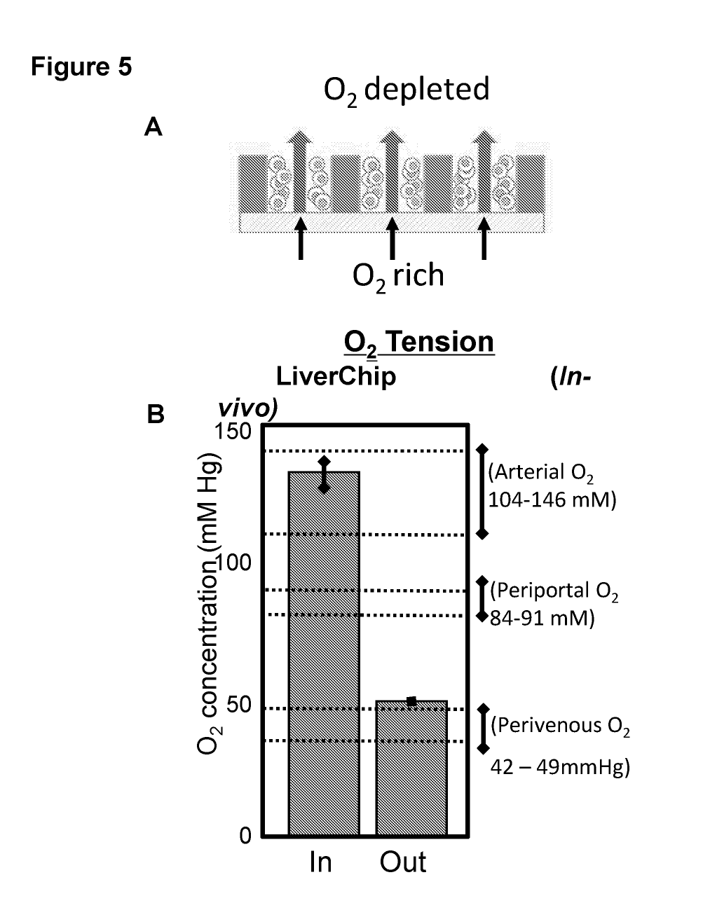

FIG. 5 shows the liver tissue model recreates an approximation of the physiological oxygen gradient. FIG. 5A is a schematic of the liver tissue model showing O₂ tension. FIG. 5B demonstrates O₂ tension at a flow rate of 0.25 mL/min. Oxygen is a key regulator of cell survival and function. For example, expression of key drug metabolizing enzymes (P450s) is regulated by O₂ tension.

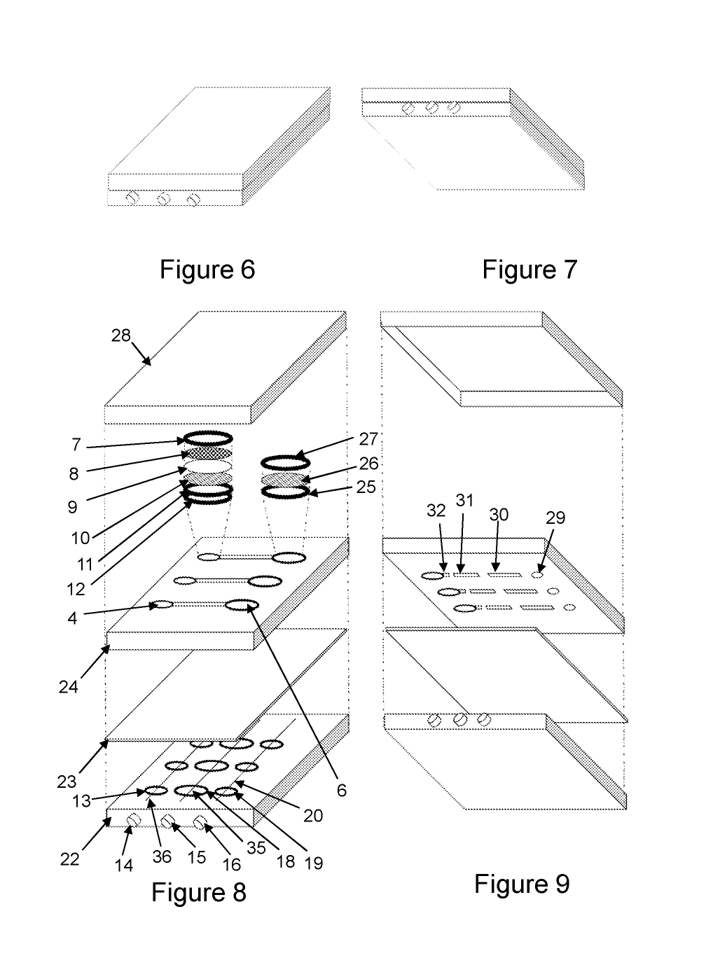

FIG. 6 is an isometric view (top) of an array of three perfused bioreactors in multiwell plate format.

FIG. 7 is an isometric view (bottom) of an array of three perfused bioreactors in multiwell plate format.

FIG. 8 is an exploded view (top) of an array of three perfused bioreactors in multiwell plate format. For simplicity, only a 3-unit bioreactor array is shown.

FIG. 9 is an exploded view (bottom) of an array of three perfused bioreactors in multiwell plate format.

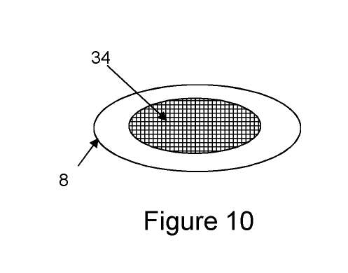

FIG. 10 is an isometric detail view of the scaffold of a perfused bioreactor.

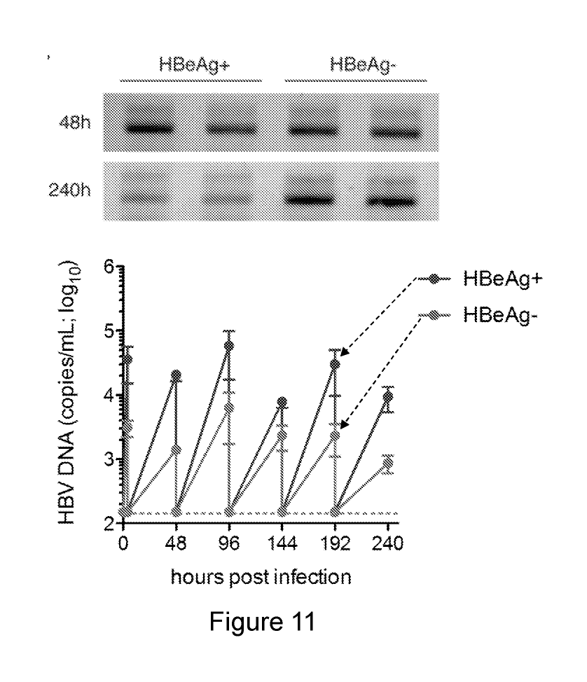

FIG. 11 shows longitudinal HBV DNA copies in the supernatants of the liver tissue model cultures with adjustment to compensate for supernatant removal. This representation more accurately represents that the data points are a 48 hour accumulation interval rather than just being an accumulation only over the complete kinetic. cccDNA is measured in liver tissue model cultures from both HBeAg+ and HBeAg− patient isolates.

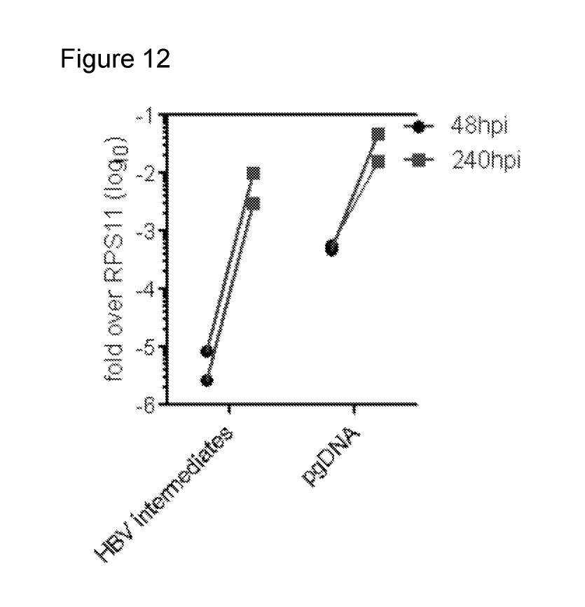

FIG. 12 demonstrates the expression of HBV replication intermediates and pgRNA in the liver tissue model at two time-points, at 48 and 240 hours post infection. HBV infection of human hepatocytes in the liver tissue model leads to the accumulation of HBV replication intermediates inside the cells. In addition to replication intermediates, pregenomic (pg) RNA is readily detectable and increases over the course of infection.

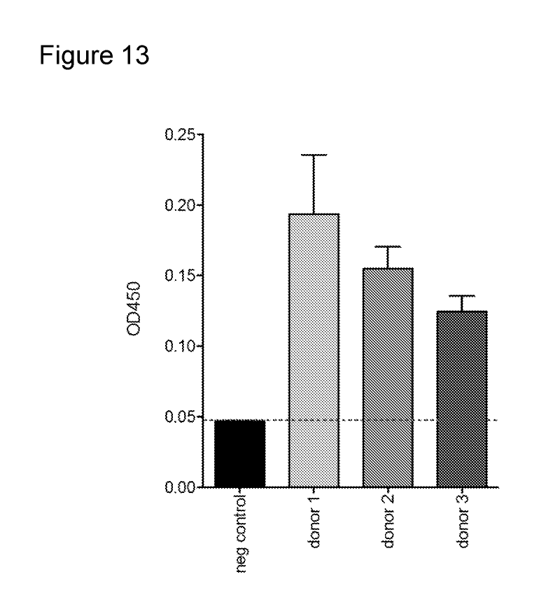

FIG. 13 shows virus replication in the liver tissue model as evidenced by secretion of HBsAg. Different hepatocyte donors are shown to exhibit similar HBsAg secretion levels following infection with HBeAg+ HBV.

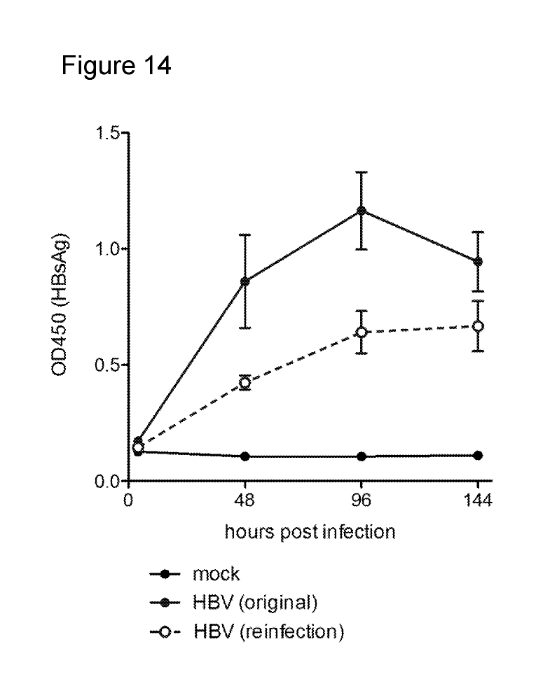

FIG. 14 shows supernatants from the liver tissue model are infectious in vitro. Supernatant extracted from a previous experiment (FIG. 11, day 10 sample) is infectious in reinfection experiments (HBsAg Elisa). HBsAG levels for hepatocytes infected with patient serum (original) are compared with hepatocytes infected with supernatant extracted from an liver tissue model infection experiment (FIG. 11) using the same patient serum (reinfection).

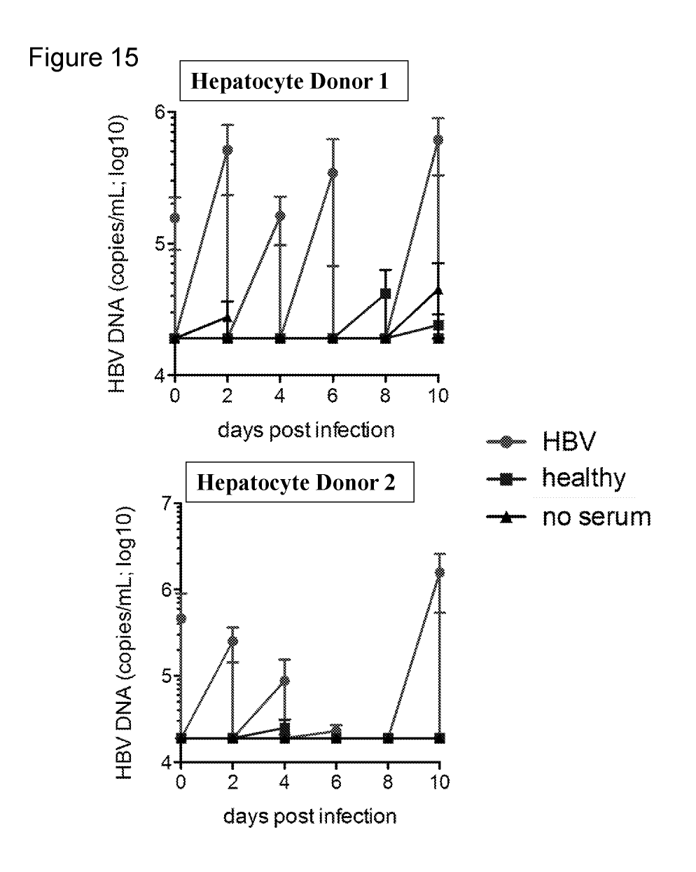

FIG. 15 shows multiple human hepatocyte donors can be utilised to evaluate HBV infection using the liver tissue model. Infection results in comparable HBV DNA levels in multiple hepatocyte donors (Donor 1 and Donor 2).

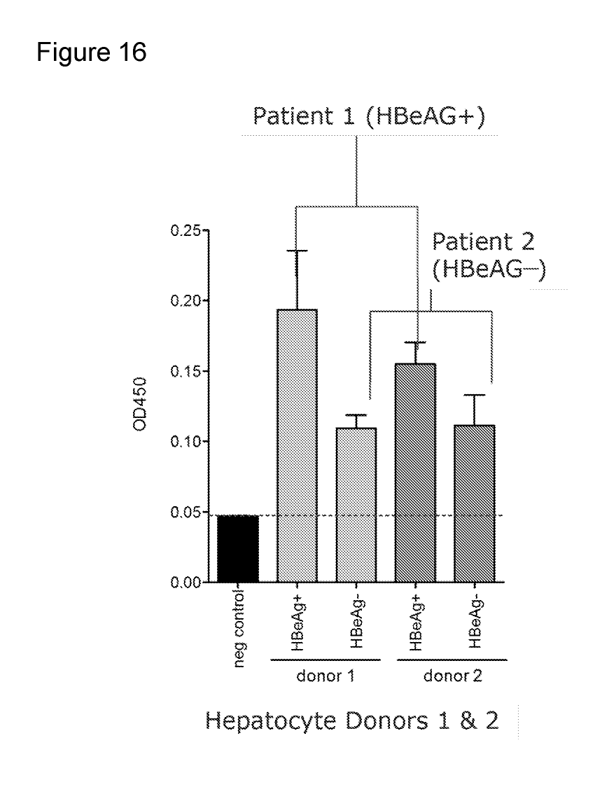

FIG. 16 shows multiple human hepatocyte donors can be utilised to evaluate HBV infection using the liver tissue model. Infection results in comparable HBsAg levels in multiple hepatocyte donors. Donors from male, female, adult and juvenile can be infected.

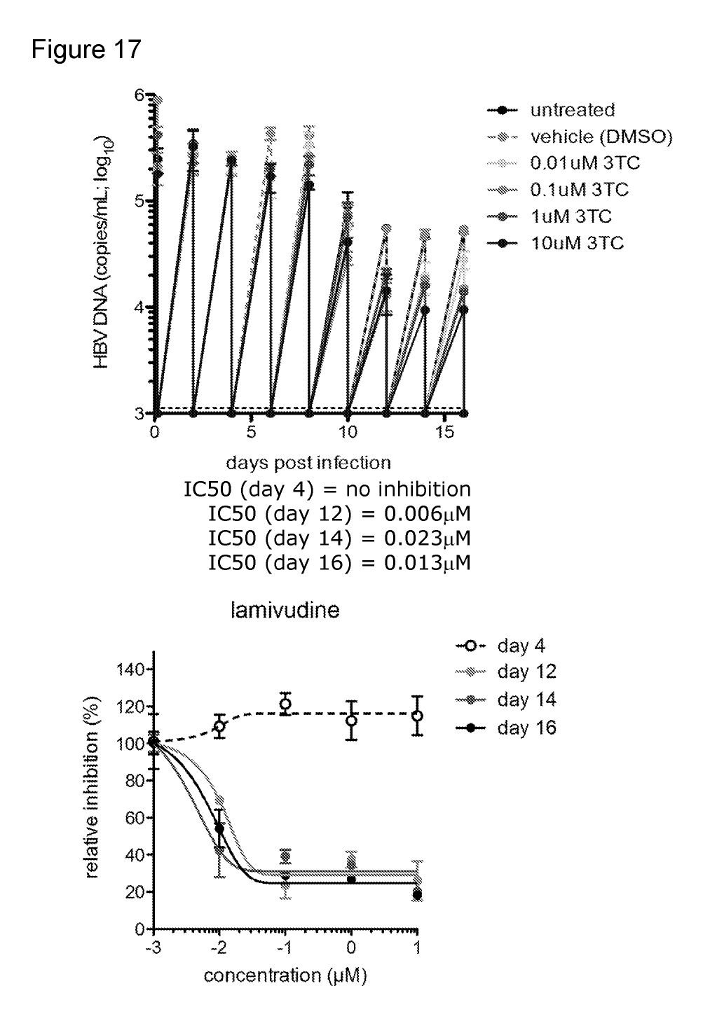

FIG. 17 shows that the liver tissue model can be used to evaluate drug candidates against HBV. Lamivudine (3TC) is dosed at various concentrations every 48 hrs and is shown to inhibit HBV replication. IC50s are shown which are comparable to in vivo treatment with Lamivudine.

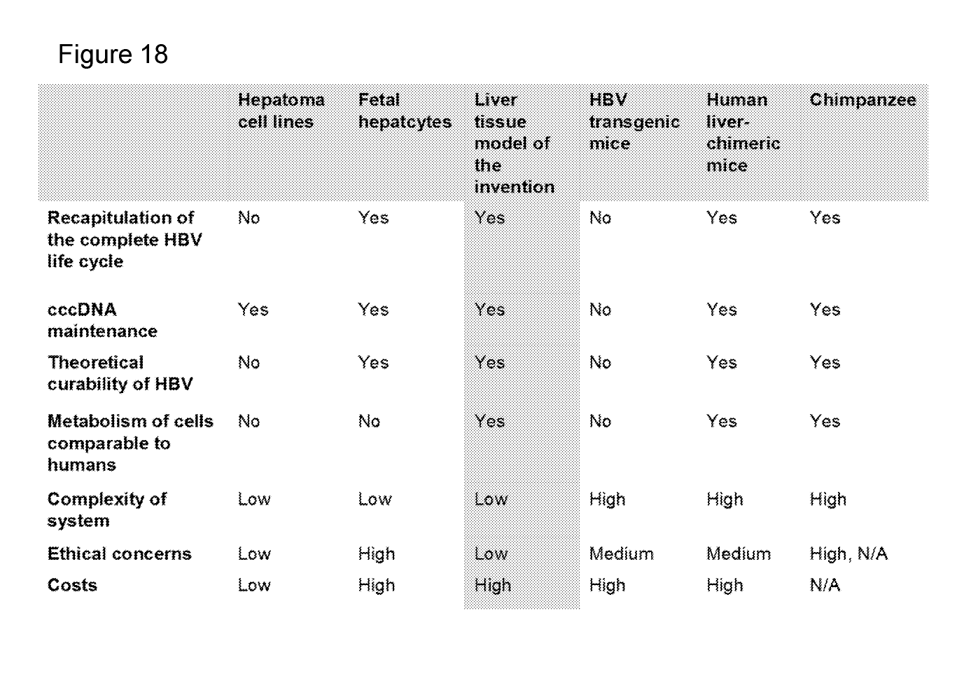

FIG. 18 shows a comparison of the liver tissue model of the invention and available HBV model systems.

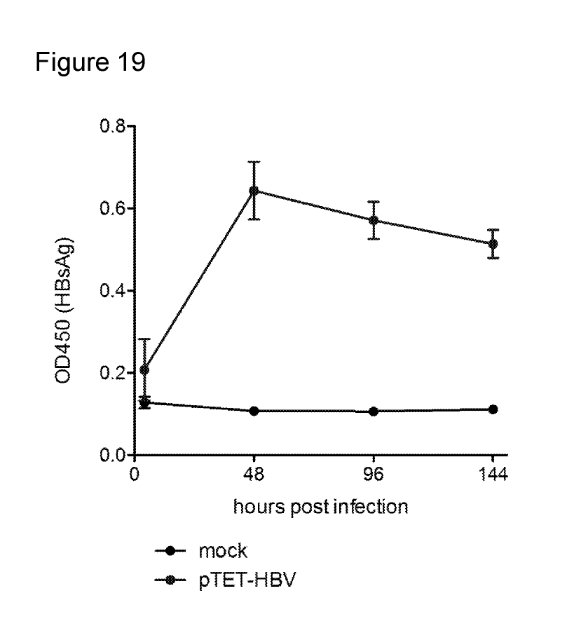

FIG. 19 shows HBV vector derived from plasmid transfections in HepG2 cells is infectious in liver tissue model cultures.

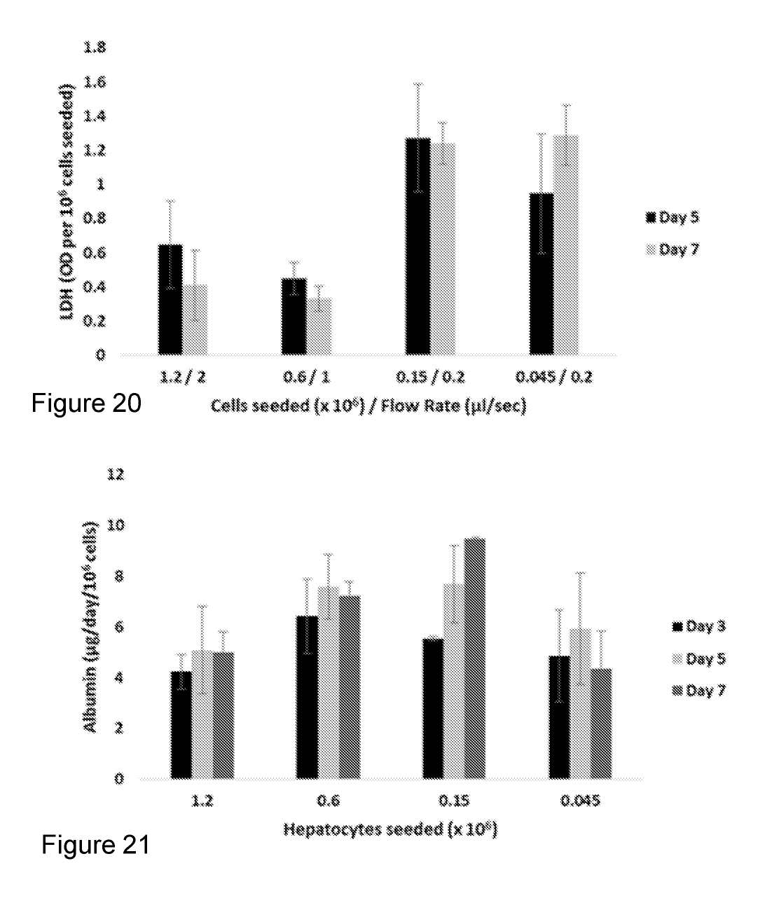

FIG. 20 shows LDH release from cells cultured in the liver tissue model at several seeding densities and corresponding flow rates (See Table 1). Increase in LDH is indicative of necrotic cell death.

FIG. 21 shows albumin secretion from cells cultured in the liver tissue model at several seeding densities and corresponding flow rates (See Table 1).

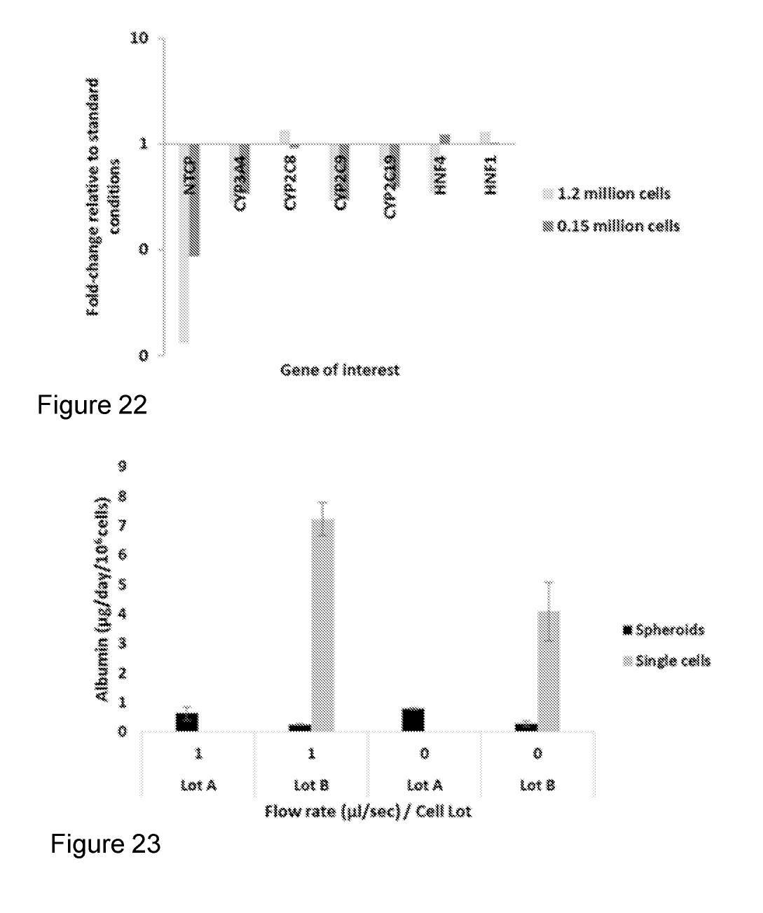

FIG. 22 shows the fold change of expression of several genes of interest relative to optimum conditions (0.6×10⁶ cells per scaffold at flow rate of tpliscc). Note: RNA isolated from 0.045 million cells did not yield high enough quality RNA for qPCR. NTCP is involved in the entry mechanism of HBV virus.

FIG. 23 shows albumin secretion from human hepatocytes seeded as single cells or spheroids at Day 7. Spheroids were formed and seeded for 2 different lots of hepatocytes. Albumin secretion from hepatocytes indicates that the cells are in a differentiated state.

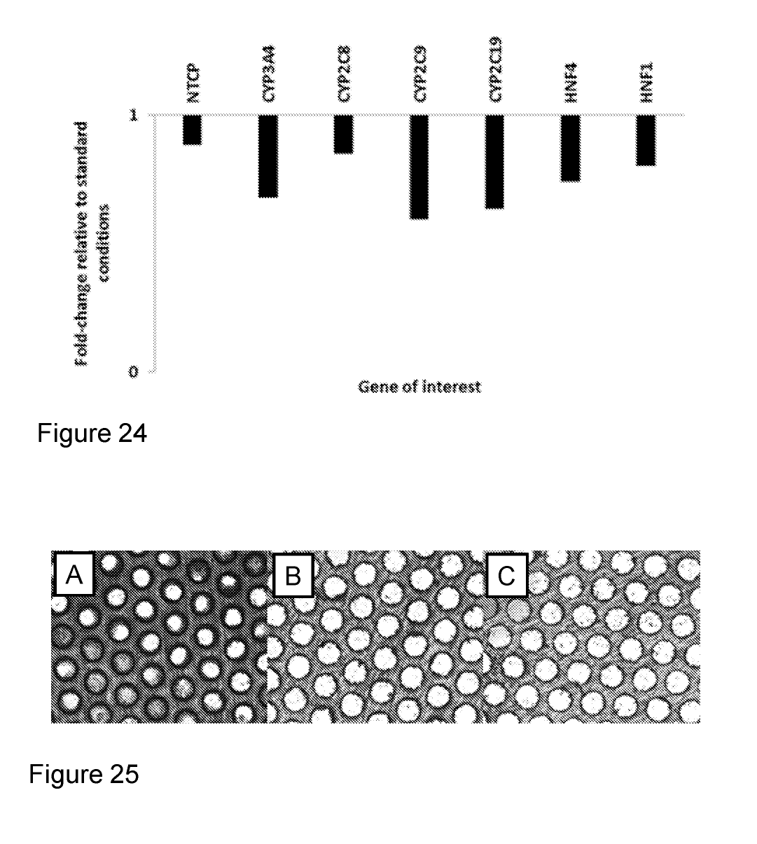

FIG. 24 shows a fold change of several genes of interest of 0.6 million cells seeded without flow relative to optimum conditions (0.6 million cells seeded with flow). Note: RNA isolated from all spheroids formed did not yield high enough quality RNA for qPCR. Poor quality RNA is a result of a poor culture of hepatocytes. NTCP is involved in the entry mechanism of HBV virus. Gene expression of NTCP is downregulated in all conditions in comparison with optimum conditions. Down-regulation of hepatic phenotypic markers such as the genes measured indicated a hepatocyte in a de-differentiated state.

FIG. 2A shows a phase contrast image of the scaffold, with 0.6 million cells seeded with flow. FIG. 2B shows a phase contrast image of the scaffold, with 0.6 million cells seeded in spheroids with flow. FIG. 2C shows a phase contrast image of the scaffold, with 0.6 million cells seeded in spheroids without flow.

Normal histoarchitecture is rapidly lost in standard culture and the interactions between multiple cell types is central in many disease processes. However, the 3D perfusion culture in the liver tissue model described herein maintains functional histoarchitecture over time, including sinusoids, cell interactions and polarity.

Liver tissue model set up, cell seeding, medium change and infection with hepatitis A, B, C, D or E virus and other liver cell infective viruses.

The following experiments should be performed in a class III laboratory and may be applicable to hepatitis viruses, cytomegaloviruses, adenoviruses and other viruses infective to human liver cells.

Materials

Media

Williams E medium for seeding and maintenance media suitable for the long term culture of human hepatocytes.

Williams E medium for the infection of cells with the virus. An alternative media can be Dulbecco's Eagle Medium, or other suitable media.

Liver tissue model components: fluidic plate, pneumatic plate, ECM coated scaffolds (13; sterile), retaining rings (13), filters (13), sterile pusher, sterile tweezers, multiwell plate lids (sterile), membranes (sterile), screws (43) and washers (43).

Virus infection source is from human blood products from patients infected with virus. Alternative virus sources may be used. The virus can be purified and concentrated e.g. by precipitation to ensure sufficient concentration of the viral particles for adding to the liver tissue model. Other sources can include viral particles from transgenic animal models that produce human viruses. Viral DNA may also be introduced through transfection of plasmids containing the viral genome which are transcribed and translated to viral particles.

The liver tissue model and methods of the invention may make use of the following commercially available media:

“Seeding Media” CM3000—Thawing/Plating Supplement Pack (Invitrogen)

For use with: Williams E Medium (WEM, A1217601)

Applications: Thawing and plating cryopreserved hepatocytes, plating fresh hepatocytes

Contents: (concentrations in parenthesis)

Fetal Bovine Serum

Dexamethasone in DMSO (10 mM)

Thawing/Plating Cocktail—A, containing (18 mL total volume):
5.0 mL Penicillin/Streptomycin (10,000 U/mL/(10,000 μg/mL)
500 μL Human Recombinant Insulin (4 mg/mL)
5.0 mL GlutaMAX™ (200 mM/100×)
7.5 mL HEPES, pH 7.4 (1M)

Directions for supplementing medium *:

To 500 ml medium, add:
Entire contents of Fetal Bovine Serum tube (25 mL)
50.0 μL Dexamethasone in DMSO
Entire contents of Thawing/Plating Cocktail—A (18 mL)

“Cell Culture Media” CM4000—Cell Maintenance Supplement Pack (Invitrogen)

For use with: Williams E Medium (WEM, A1217601)

Applications: Incubation medium for cells in suspension, maintenance of cultured hepatocytes

Contents: (concentrations in parenthesis)

Dexamethasone in DMSO (10 mM)

Cell Maintenance Cocktail—B, containing (20 mL total volume):
2.5 mL Penicillin/Streptomycin (10,000 U/mL/(10,000 μg/mL)
5.0 mL ITS+
   human recombinant insulin (0.625 mg/mL)
   human transferrin (0.625 mg/mL)
   selenous acid (0.625 μg/mL)
   bovine serum albumin (BSA)-(0.125 g/mL)
   linoleic acid (0.535 mg/mL)
5.0 mL GlutaMAX™ (200 mM/100×)
7.5 mL HEPES, pH 7.4 (1M)

Directions for supplementing medium*:

To 500 ml medium, add:
5.0 μl Dexamethasone in DMSO
Entire contents of Cell Maintenance Cocktail—B (20 mL)

Liver Tissue Model and Apparatus

A system has been developed based on a perfused scaffold with microchannels and utilising parallel means for circulating cell culture medium through the microchannels of liver tissue, making the technology exceedingly suitable for liver infection studies.

The system has as an array of perfusion bioreactor and reservoir pairs for cell or tissue culture, and valves and pumps actuated in parallel via common control channels and re-circulating medium through the array of bioreactor and reservoir pairs. Each bioreactor of the array includes a bioreactor well and its own reservoir well. The bioreactor wells and reservoir wells are connected by fluidic channels allowing re-circulation of cell culture medium.

Each bioreactor/reservoir pair is fluidically isolated from all other bioreactor/reservoir pairs in the array. The valves and pumps of all bioreactors in the array are actuated in parallel via common hydraulic or pneumatic control channels.

The bioreactor/reservoir pairs are fabricated or microfabricated in the fluidic manifold. The control channels are fabricated or microfabricated in the control manifold. Diaphragm valves are created by sandwiching a monolithic polymeric membrane between fluidic and control manifolds.

The membrane between the control and fluidic channels can be deflected by hydraulic or pneumatic actuation applied through the control channels. Cell culture medium in multiple bioreactors is pumped by sequential actuation of the valves connected in series.

Each bioreactor includes a well including a three-dimensional cell/tissue support structure/scaffold. The cell scaffold or carrier is made out of a synthetic or natural porous material. The cell scaffold is formed by an array of microchannels in a solid film or sheet supported by a microporous filter or membrane. The scaffolds can be removed from the bioreactor wells. All bioreactors/reservoir pairs in the array are covered by a common removable lid, and cell/tissue seeding, agent addition, or sample collection can be added by pipetting or robotics.

Representative of the liver tissue model apparatus for use herein, the array for simplicity includes only three bioreactor/reservoir pairs in the multiwell plate format as shown in FIGS. 6-10. However, the size of the components can be easily scaled-down and a considerably higher number of bioreactor/reservoir pairs can be placed on a single plate. The scaffolds are accessible from the top. The main functional component of each bioreactor is a well 4 with a 3D cell/tissue holding scaffold or carrier 8. The design includes rims in the wells to reduce meniscus of the fluid surface and thereby minimizing the optical distortion during cell/tissue observation under a microscope; and making the reactor/reservoir pairs in the chimney arrangement to minimize cross-contamination between the adjacent reactors. The chimneys can be matched with rings of the corresponding shape in the lid to minimize evaporation of the fluid from the reactor wells, reservoir wells, and the connecting surface channels. Making the valves in the clamshell shape compensates for a stretched or wrinkled membrane and improves the valve performance. Making the valves and pumps of an oblong shape reduces areas where air bubbles can be trapped.

The scaffold or carrier can be made using conventional silicon processing technology, such as photolithography, wet etching, or deep reactive ion etching; micromachining; electro-discharge machining; reaction injection molding; thermoplastic injection molding; micromolding; punching; any of the solid free form technologies, such as three dimensional printing; or other types of manufacturing which can create micro through-holes in sheets of material, especially manufacturing technologies for plastics, such as micromolding, embossing, laser drilling, or electron beam machining. Molds for some of these processes can be made using methods such as lithography and micromachining, electro-discharge machining, and electroplating.

A number of materials are commonly used to form a matrix. Unless otherwise specified, the term “polymer” will be used to include any of the materials used to form the matrix, including polymers and monomers which can be polymerized or adhered to form an integral unit, as well as inorganic and organic materials, as discussed below. In one embodiment the particles are formed of a polymer which can be dissolved in an organic solvent and solidified by removal of the solvent, such as a synthetic thermoplastic polymer, either biodegradable or non-biodegradable, such as polyesters, polyurethanes, polystyrene, polycarbonates, ethylene vinyl acetate, poly (anhydrides), polyorthoesters, polymers of lactic acid and glycolic acid and other a hydroxy acids, and polyphosphazenes, protein polymers, for example, albumin or collagen, or polysaccharides. Examples of non- polymeric materials which can be used to form the matrix include organic and inorganic materials such as hydoxyapatite, calcium carbonate, buffering agents, and lactose, as well as other common excipients used in drugs, which are solidified by application of adhesive or binder rather than solvent. In the case of polymers for use in making devices for cell attachment and growth, polymers are selected based on the ability of the polymer to elicit the appropriate biological response from cells, for example, attachment, migration, proliferation and gene expression.

Other suitable polymers can be obtained by reference to The Polymer Handbook, 3rd edition (Wiley, N.Y., 1989).

Referring to FIGS. 6-10, the cell/tissue support structures 8 can be formed, for example, by a porous membrane or by an array of microchannels in a solid film or sheet supported by a microporous filter 9. The porous scaffolds can be fabricated, for example, by fiber bonding (Vacanti, et al. MRS Proceedings, vol. 252 (1992); Mikos, et al., J. Biomed. Mater. Res. 27: 183-189 (1993), solvent casting/particulate leaching (Mikos, et al., Biomaterials 14: 323-330 (1993), gas foaming (Mooney, et al., Biomaterials 17:1417-1422 (1996), and gas separation (Lo, et al., Tissue Engineering 1:15-28 (1995). Silicon scaffolds with an array of microchannels can be microfabricated using appropriate technologies such as by deep reactive ion etching technique (Powers, et al., Biotechnology and Bioengineering, 78:257 (2002). Polymer scaffolds with microchannels can be produced by laser- micromachining (Brenan, et al., Proc. SPIE, 3912, 76-87, Prog. Biomed. Optics, Micro- and Nanotechnology for Biomedical and Environmental Applications, San Jose, Jan. 26-27, 2000), injection molding (Weibezahn, et al., Micro System Technologies '94, H. Reichl and A. Heuberger, eds., vde-verlag gmbh, Berlin, pp. 873-878) or photopolymerization.

The solid scaffold 8 with microchannels can have a top layer containing an array of microchannels holding the cells. Each microchannel in the array is the functional unit of the bioreactor. Under this layer, there is a microporous membrane or a filter 9. The membrane can be a monolithic part of the cell holding scaffold or it can be e.g. thermally or ultrasonically bonded to it. The cell and/or tissue holding scaffold can be provided with sealing gaskets 7, 11, a support scaffold 10, and an insert 12. The microchannels 34 can have square, slit, circular, elliptical or oval cross-section. The typical size of the channels is several hundred microns. The slits can be from several hundred microns to several millimeters long. The scaffold thickness is typically several hundred microns.

Both the fluidic 24 and control 22 manifolds can be fabricated e.g. by micromechanical milling out of polymers such as polycarbonate. This can be cost effective in the small batch fabrication. In large volume fabrication, mass replication techniques such as injection molding and materials e.g. polystyrene or polycarbonate can be used. Alternatively 3D printing techniques may be used. The membrane material can be e.g. polydimethylsiloxane (PDMS). The membrane 23 can be e.g. bonded to the fluidic and control manifold by plasma oxidizing the mating surfaces and immediately pressing the parts together (Duffy et al., Anal. Chem., 70, 4973-4984 (1998). Alternatively, the membrane 23 can be sandwiched between the fluidic 24 and control 22 manifolds e.g. by means of a fastening or latching mechanism providing a constant force on the membrane and holding the manifolds together.

The scaffold 8 can be e.g. press-fitted into the lower tapered section of the bioreactor well 4 in the fluidic manifold 24. The bioreactor well 4 is connected with the reservoir well 6 by two fluidic channels. The upper e.g. U-shaped channel 5 is used to return the cell culture medium from the bioreactor well 4 into the reservoir well 6. The bottom part of the reservoir can contain a face-off for inserting a microporous filter 26 with a filter support 25 secured in the reservoir well by a press-fitted insert 27. Bioreactor/reservoir pairs in the array on the plate are identical. The filter 26 can be used to remove cell debris from the cell culture medium. Using the filter in the reservoir well can improve reliability of the valves and pumps. In addition, it can eliminate clogging the membrane or microporous filter 9 on the backside of the cell/tissue holding scaffold 8. Filtered culture medium is sucked through port 29 into the bottom fluidic channels 30, 31, and 32 connecting the reservoir 6 and bioreactor wells 4. The channel is provided with three diaphragm valves forming a pump. The valves are created by sandwiching a monolithic elastomer membrane 23 between fluidic 24 and control 22 manifolds. A valve is created where a control channel crosses a fluidic channel. The valves can be e.g. normally closed. In that case, by applying vacuum to the channels 20, 35, and 36 in the control manifold through the fittings 16, 15, and 14 sealed with O-rings, the elastomer membrane 23 is deflected down, the valves are opened, and cell culture medium fills the valve displacement chambers 19, 18, 13 and all other connected displacement chambers above the membrane 23. Applying positive pressure forces the membrane against the valve seats and the cell culture medium out of the displacement chambers of the valves. The valves of each pump are operated in a six-step cycle. Initially, all valves are closed.

In the first step, the inlet valve 19 and all other valves in bioreactor/reservoir pairs connected in series by the control channel 20 are opened.

In the second step, the main diaphragm valve 18 and all other valves in bioreactor/reservoir pairs connected in series by the control channel 35 are opened. In the third step, the inlet valve 19 and all other valves in bioreactor/reservoir pairs connected in series by the control channel 20 are closed. In the forth step, the outlet valve 13 and all other valves in bioreactor/reservoir pairs connected in series by the control channel 36 are opened. In the fifth step, the main diaphragm valve 18 and all other valves in bioreactor/reservoir pairs connected in series by the control channel 35 are closed. In the sixth step, the outlet valve 13 and all other valves in bioreactor/reservoir pairs connected in series by the control channel 36 are closed. The valves pumping the cell culture medium can be controlled e.g. by solenoid valves connected to sources of vacuum and pressurized air.

The normally closed monolithic membrane valves are self-priming and pump cell culture medium forward or backward simply by reversing the actuation cycle. By adjusting the volume of the diaphragm valve displacement chamber, the volume pumped per actuation can be determined at the design stage. Therefore, diaphragm pumps may be used to precisely meter the volumes of cell culture medium. If the displacement chambers of the pumps are identical, the cells/tissues in all bioreactors will be perfused at the same flow rate. In contrast, if the pumps have different volumes of displacement chambers, the cells/tissues in each bioreactor can be perfused at different flow rates.

The bioreactor and reservoir pairs are primed e.g. by manual or robotic pipetting of cell culture medium into the bioreactor or reservoir well and activating the pumping cycle in forward or reverse direction. If it is necessary to remove air bubbles from the fluidic channels, air bleeding ports fitting a screw and a sealing O-ring can be used (not shown). The air bleeding ports may be connected with the bioreactor well 4 via a channel.

Referring to FIGS. 6-10, cells are seeded into the scaffolds 8 by dispensing (e.g. by manual or robotic pipetting) cell suspension into the bioreactor wells 4. Cell culture medium is circulated from the reservoir well 6 into the bioreactor well 4. After perfusing the 3D cell culture in the scaffold 8 in the bioreactor well 4, the cell culture medium is returned to the reservoir well 6. Each bioreactor of the array has its own reservoir 6 and its microfluidic channels 5, 30, 31, and 32 are completely isolated from all other bioreactors in the array. Cell culture medium is re-circulated using diaphragm pumps. Three diaphragm valves connected in series form a diaphragm pump. The valves 19, 18, and 13 and therefore the pumps are created by sandwiching a monolithic elastomer membrane 23 between fluidic 24 and control 22 manifolds. A valve is created where a control channel crosses a fluidic channel. The thin membrane 23 between the control and fluidic channels can be deflected by hydraulic or pneumatic actuation applied through the control channels. Cell culture medium is pumped by sequential actuation of the valves connected in series. The valves of all bioreactors/reservoir pairs in the array are actuated in parallel via common hydraulic or pneumatic control lines. As a result, in this case five completely isolated perfusion bioreactors can be addressed by three common pneumatic or hydraulic lines. However, the number of bioreactor/reservoir pairs in the array operated by three pneumatic or hydraulic lines can be considerably scaled up.

The valves and pumps are scalable and can be microfabricated in dense arrays. Unger, et al., Science 286, 113 (2000), T. Thorsen, et al., Science 298,580 (2002), and W. H. Grover, et al., Sensors and Actuators B 89, 314 (2003), describe processes of producing monolithic valves and pumps.

Due to the fact that the array of bioreactor/reservoir pairs has an open design and is covered by a common removable lid 28, cell seeding as well as active agent addition and sample collection can be performed using automated robotic workstations.

Flow rates through the system are determined by the cellular metabolic needs and by mechanical stress issues. Flow rates in the range of 0.1-1 microliter/min of medium per 1000 cells are required on a near-continuous basis (short periods of up to 15 min of no flow are feasible).

Each bioreactor typically contains from 500 to 50,000 cells, or alternatively up to 2,000,000 or 5,000,000 cells depending on the type of assay being performed. The design of the scaffold allows the system to be scaled very readily in units of about 2000 (i.e., one channel). The flow rates through the system might be varied during the time of culture or assay in order to perform the assay (e.g., flow rates might be slowed to allow complete conversion of a compound, or increased in order to keep a constant concentration of the compound).

Sensors can be used to detect changes in pH, oxygen levels, specific metabolites such as glucose, presence or absence of an indicator molecule such as a viral protein, or any other indicia of an effect on the tissues or a material exposed to the tissues within the bioreactor.

Readouts of injury or infection can be based on changes in fluorescence of the tissue as detected by a miniaturized fiber optic array which excites fluorescence via either single or multiphoton means. The nature of the excitation is a critical parameter. Multiphoton excitation offers several advantages over single photon, in terms of resolution and prevention of tissue damage.

Many types of fluorescent readouts are possible. Changes in basic metabolic parameters of the tissue can be assessed by measuring the change in NAD(P)H levels via intrinsic fluorescence of these molecules. Cells can also be pre-loaded with a dye which leaks in the case of membrane damage, resulting in a decrease of fluorescent intensity. Alternatively or in addition, reporter genes can be transfected into the cells under the control of a stress- related promoter which is activated during tissue injury to produce a fluorescent product. This latter approach is of particular interest for detecting viral infection on a rapid time scale.

A panel of potential indicators which will vary in either fluorescence intensity and/or spectrum have been identified. Since responses may require monitoring cellular biochemical state within normal tissue structure, it may not be sufficient to analyze only the surface layer of cells in the tissue, but to selectively monitor the cellular strata several cells deep into the channel interior. These requirements can be summarized into four design criteria for the optical detection system: (1) depth selection detection in thick (300 μm) tissue, (2) flexible excitation and detection scheme to image a variety of indicators, (3) minimally invasive to the living tissue culture in the device, (4) fast signal detection with high sensitivity, (5) rugged and field adaptable.

Using single photon excitation, confocal detection is used to separate fluorescence which originates from the channel interior from its surface. A confocal microscope is a well-developed instrument designed to optically section thick specimens. Two apertures or pinholes are arranged in conjugate planes; one in front of the light source and one in front of the detector. This design can be simplified and made more robust for on-line detection by the use of single mode fiber optics. Through a dichroic beam splitter, excitation light is introduced into a single mode fiber (beam splitter is not depicted). The light emitted from the fiber can be collimated by a lens. A second lens can focus the collimated light into the channel of the tissue chip. High resolution is not critical in this application (no imaging is required) and thus low optics and the chip to provide spaces for the hydraulic design in the flow chamber. The fluorescence from the sample is collected by two relay lenses and reflected back into the single mode fiber. The small diameter fiber functions simultaneously as the excitation and emission pinhole aperture in this system. Fluorescence originated outside the focal region can not be refocused by the relay lenses on to the fiber optics and is rejected. This process provides us depth discrimination. A number of chromophores with excitation wavelengths spanning near-UV to the blue-green region of the spectra can be considered. Fluorescence indicators of particular interest are the endogenous chromophores, pyridine nucleotides. The pyridine nucleotides, NAD(P)H are excited in the region 365 nm and fluoresce in the region 400-500 nm. Another indicator of interest is green fluorescence protein (GFP) which can be excited in either near UV or the blue region of the spectrum and typically emits at about 510 nm. In order to excite this wide range of chromophores, a tunable UV argon-ion laser can be used.

Chromophores can be excited by the simultaneous absorption of two photons each having half the energy needed for the excitation transition. Since the two-photon excitation occurs only at the focal point of a high numerical aperture objective, a region of high temporal and spatial concentration of photons. Using two-photon excitation, over 80% of the total fluorescence intensity comes from a 1 μm thick region about the focal point for a 1.25 numerical aperture objective. This depth discrimination effect of two-photon excitation arises from the quadratic dependence of two-photon fluorescence intensity upon the excitation photon flux which decreases rapidly away from the focal plane. The depth discrimination is a result of the physics of the excitation method and no confocal detection pinhole aperture is needed. This localization of two-photon excitation can be best visualized in a simple bleaching experiment.

To demonstrate the effect of two photon excitation, a two photon excitation volume was focused in the center of a 15 μm fluorescent latex sphere. The excitation volume was scanned repeatedly along the x axis until photobleaching occurred. A 3-D image stack of the latex sphere was acquired, in which a series of images are x-y planes of the sphere at increasing distance from the center. No photobleaching was observed beyond 1 μn.

Two-photon excitation allows selective assessment of the tissue physiological state at any point in the interior of the tissue chip channel.

There are a number of advantages to the multi-photon approach as compared with confocal approach where the sample's absorption and scattering coefficients are high, such as those in tissues: (1) The typical scattering and absorption in the infrared spectral range is over an order of magnitude less than the near UV or the blue-green region. Using infrared excitation in the two-photon microscope minimizes the attenuation of the excitation signal.

(2) Confocal microscopy uses the emission pinhole aperture to reject out of focus light. Inside deep tissue, scattering of the signal photons is inevitable.

The consequent path deviation results in a significant loss of these photons at the confocal pinhole. The collection geometry for the fluorescence photons is less critical in the two-photon case where a large area detector can be used without a pinhole aperture. Most of the forward-scattered photons can be retained. (3) Two-photon excitation minimizes tissue photo-damage.

Conventional confocal techniques obtain 3-D resolution by limiting the observation volume, but fluorescence excitation occurs throughout the hourglass-shaped light path. In contrast, two-photon excitation limits the region of photo-interaction to a sub-femtoliter volume at the focal point. (4) Two-photon excitation wavelengths are typically red-shifted to about twice the one-photon excitation wavelengths. This wide separation between excitation and emission spectrum ensures that the excitation light and the Raman scattering can be rejected while filtering out a minimum of fluorescence photons. (5) Many fluorophores have found to have very broad two-photon absorption spectra. A single properly-chosen excitation wavelength can excite a wide range of fluorophores with emission bands ranging from near-UV to near-infrared.

Sensors other than fluorescent sensors can also be used. For example, samples can be analyzed by using infrared spectrophotometers, ultraviolet spectrophotometers, gas chromatograms, high performance liquid chromotograms, mass spectrometry, and other detection means known to those of skill in the art. These can be used to measure nutrients, gases, metabolites, pH, and other indicators of cell activity, infection, and metabolism. Measurements may be made on the cells themselves or on the culture medium, or both. Measurements may be made as a time course assay or an end-point assay or both during culture and at the end of culture.

Experimental Protocol

Components to be autoclaved: Fluidic plate, filters, retaining rings, tweezers, pusher, screws and washers.

Sterile (γ-irradiated) components include membrane and scaffolds. The scaffold can be extracellular matrix coated in order to aid cell adherence.

Liver Tissue Model Set Up

Assemble the reactor by placing a membrane on the pneumatic plate and putting the fluidic plate on top. Screw together with 43 screws and washers, starting in the centre and working outwards (only one screw required in the centre of the middle row) using a calibrated torque driver set to give a torque on each screw of 30 Ncm.

Prime the system with warm seeding medium by adding 450 μl to the reservoir side.

Flow media up for 2.5 min at 2.0 μl/second until fluid comes through the filter support and bubbles are eliminated.

Fill reactors with a further 1.4 ml seeding medium to cover the surface channel. This media may contain infective source for infection of cells during seeding.

Add filters, scaffolds and retaining rings and push into place.

Leave in the incubator until seeding (with/without flow).

Cell Seeding
a) Freshly-isolated or cryopreserved human hepatocytes, thawed according to the vendors instructions, are seeded into the scaffold at a density of approximately 600,000 cells per well. This number may vary depending on each individual lot of cells, and number of microchannels, and is determined empirically.
b) Count cells and adjust volume to 6×10⁶ cells per mL for a seeding volume of 100 μl per well.
c) Remove primed plate from the incubator, stop flow and remove medium down to the retaining ring, add 400 μl seeding medium to each well prior to seeding cells.
d) Add cells to each well and start flow pumping down at 1 μl/sec.
e) Add 900 μl seeding medium to achieve a total volume of 1.4 ml (with a further 200 μl in the channels between the plates
f) Move plates to incubator with flow pumping down at 1.0 μl/sec through the scaffolds for 8 hr. Flow will automatically reverse to pump up through the scaffolds after 8 hr.
g) Change from seeding medium to culture medium after 24 hr. This media may contain virus infection source as appropriate to the experiment.

Infection/Transfection

Infected patient serum or other whole virus source may be added at stage (e) above or at a subsequent media change when tissues have formed on the scaffold as appropriate to the experiment. Such source may be added to the medium to constitute up to ideally no more than 10% of the media volume (although a higher concentration is not excluded) or may be diluted in medium up to 10,000-fold prior to addition to the wells.

Viral infection may alternatively be performed prior to seeding into the scaffold at stage (b) above using infected patient blood product, which may be concentrated to increase the concentration of viral particles or diluted to achieve the highest efficiency infection. Infection may proceed through wholly physiological mechanisms or enhanced through the addition of factors reported to promote entry of virus, for example the addition of PEG. Cells may also be transfected with plasmid DNA at this time using any one of a number of standard protocols for transfection well known in the art.

Virus infection source may be incorporated at any media change and may be a single addition or multiple additions at different media changes.

Medium Change

Medium is exchanged after the first 24 h and each 48 h thereafter for the period of culture, which may be more than 14 days.

At each media change, hepatitis infected human serum or other source of virus may be added the medium to constitute up to 10% of the media volume. Viral source may be undiluted or may be diluted in medium up to 10,000-fold prior to addition to the wells. Stop flow and aspirate medium from above scaffold, channel and reservoir. Remove media to level of the retaining rings. Care must be taken not to remove total medium from above the scaffold to avoid air bubbles forming.

Place 400 μl medium in each well and run flow down for approx. 3.5 min at 1.0 μl/sec while continuously aspirating medium from the reservoir and channel.

Replace with 1.4 ml medium and return to incubator.

Verification

Hepatitis replication may be confirmed by quantification of viral DNA or RNA, as appropriate to the strain of hepatitis, in the culture medium.

Viral replication may be monitored by strand-specific RT-PCR assay.

Hepatitis surface antigen may also be detected in the media by Enzyme linked Immunosorbent assay (ELISA kit).

Maintenance of the liver phenotype for cell connectivity and polarity Powers et al. (2002. Tissue Engineering, Vol 8(3), pp. 499-513), which is herein incorporated by reference, shows that electron micrographs of tight junctions and desmosomes demonstrate cell-cell interactions are present and intercellular communication is enabled (see FIG. 6 of Powers et al.). Furthermore, electron micrographs show bile canaliculi structures, which demonstrates cell polarity is present. Therefore, intra-cellular interactions important in studying infection are present in the liver tissue model. Powers et al. (2002. Biotechnology and Bioengineering, Vol. 78(3), pp. 257-269), herein incorporated by reference, also shows in FIG. 9 that the channels in the scaffold are an approximation of a sinusoid and the cells form capillary bed like structures.

### EXAMPLE 1

### Hepatitis B Virus Infection of Primary Hepatocytes using the Liver Tissue Model

To demonstrate successful Hepatitis B virus infection of primary hepatocytes using the liver tissue model, HBeAg-positive and negative isolates were chosen as HBeAg+ HBV more resembles wild-type HBV as seen early during infection whereas HBeAg-negative HBV acquires mutations rendering the e-antigen non-functional. This variant of HBV is seen at later stages of infection and usually leads to much lower replication levels in the serum compared to HBeAg-positive HBV. Even though there may be some scarce reports describing culture systems for HBV using fetal hepatocytes for HBeAg-positive samples, no model has ever been described for HBeAg-negative HBV.

The incubation and sampling was provided as follows:

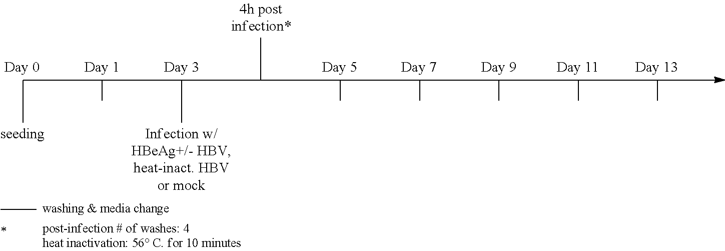

Inoculum
HBeAg-positive: 2.2e7 HBV DNA copies/mL (m.o.i.=0.75).
HBeAg-negative: 4.4e4 HBV DNA copies/mL (m.o.i=0.075).

Samples
Supernatants were sampled at each indicated time-point (n=6).
Cells were sampled at days 5 and 13 post plating (day 2 and 10 post infection, n=3).

Analytes
Supernatant for HBV DNA qPCR, Elisa for HBeAg, Cytokine production, albumin secretion.
Cells for HBV DNA qPCR, HBV cccDNA qPCR, replication intermediates by qRT-PCR.

Results

With reference to FIG. 11 longitudinal HBV DNA copies in the supernatants of the liver tissue model cultures is shown. This data representation clearly shows the difference in HBV replication between HBeAg-positive and -negative HBV. Since all the supernatant of the cultures was removed at each harvesting time point this representation accurately represents that the data points are more a 48 hour accumulation interval rather than just being an accumulation only over the complete kinetic.

With reference to FIG. 12, the expression of HBV RNA species in the liver tissue model is demonstrated. At 48 h and 240 h post-infection, cells were lysed in order to isolate total cellular RNA. Since HBV replication relies on transcription of a total of four mRNAs off the viral genome to produce viral proteins, measurement of these RNA intermediates is a definitive way to prove replication. Additionally, these mRNA intermediates do not get packaged into the virus and thus are not present in the serum used for the inoculation.

The difference between HBV intermediates and pgRNA is that the intermediates consist of only the four mRNA species used to translate the viral proteins, whereas pregenomic (pg)RNA is the complete HBV genome encoded in RNA, which is subsequently reverse transcribed by the HBV reverse transcriptase. An increase of both species by the amount seen here can only be attributed to virus replication. Therefore, HBV replication in the liver tissue model is demonstrated.

### EXAMPLE 2

### Seeding Densities and Flow Rates for the Liver Tissue Model

Method

Cryopreserved Human Hepatocytes

Cryopreserved human hepatocytes (Lot B; Triangle Research Labs USA and Lot A; Celsis IVT, USA) were prepared as per the suppliers’ protocol. The cell viability, as assessed by a Trypan Blue exclusion test, was >90%.

Cryopreserved Human Hepatocytes Cultured in the Liver Tissue Model

The hepatocytes were seeded into liver tissue model perfusion culture plates at several densities at corresponding flow rates (See Table I). Seeding medium (1.6 mL) comprising Williams E medium with plating supplements 5% FCS) (Life Technologies, UK), was provided for the first 24 h of culture.

After 24 h of seeding, seeding medium was removed and replaced with Williams' E with maintenance supplements (1.6 ml) (Life Technologies, UK) (cell culture medium). Cell culture medium was pumped in the downward direction through the scaffold/tissues at specified flow rates (See Table 1) for 8 h. After 8 h the flow direction was reversed and cells were maintained under constant flow throughout the duration of the culture. Medium was refreshed every 2 days.

The culture ended on Day 7. At each medium change (Day 3, 5 and 7), medium was stored in polypropylene Eppendorfs at −80° C. for further analysis. At the final time point, phase contrast images were taken to assess tissue morphology within the scaffolds and cells were subsequently lysed for gene expression analysis.

TABLE 1
Number of cells seeded and the corresponding flow rate.

| Cells per channel | Cells per scaffolds | Flow rate (μl/sec) |
|---|---|---|
| 4000 | 1.2 × 10⁶ | 2 |
| 2000 (optimum condition) | 0.6 × 10⁶ | 1 |
| 500 | 0.15 × 10⁶ | 0.2 |
| 150 | 0.045 × 10⁶ | 0.2* |

*0.2 μl/sec is the lowest flow rate available in the equipment used.

Spheroid Formation and Seeding in LiverChip

The hepatocytes were seeded onto non-tissue culture plastic treated 24-well plates at a density of 2000 cells per well in Williams' E medium with plating supplements (inc. 5% FCS) (Life Technologies, UK). The plates were placed on an orbital shaker at 150 rpm in a humidified incubator at 37° C., with 5% CO₂. Spheroids were formed over 72 h. Upon confirmation of spheroid formation, spheroids were seeded into the liver tissue model at a density of 0.6×10⁶ cells/well. Seeding medium (1.6 mL) comprised Williams' E medium with plating supplements (inc. 5% FCS) (Life Technologies, UK), for the first 24 h culture.

After 24 h of seeding, seeding medium was removed and replaced with Williams' E with maintenance supplements (1.6 ml) (Life Technologies, UK) (cell culture medium). Cell culture medium was pumped in the downward direction through the scaffold/tissues at 1 μl/sec for 8 h. After 8 h the flow direction was reversed and cells were maintained under constant flow of throughout the duration of the culture. Medium was refreshed every 2 days.

The culture ended on Day 7. At each medium change (at Day 3, 5 and 7), medium was stored in polypropylene Eppendorfs at −80° C. for further analysis. At the final time point, phase contrast images were taken to assess tissue morphology within the scaffolds and cells were subsequently lysed for gene expression analysis. Spheroids and single cells were seeded into LiverChip with flow as described above and compared to spheroids and single cells seeded without flow for 5 h. After 5 h without flow, flow was initiated at 1 μl/sec in the upward direction.

Endpoint Assays at Day 7

After 7 days culture, gene expression of NTCP, CYP3A4, CYP2C8, CYP2C9, CYP2C19, HNF4 and HNE1 was assessed and albumin secretion in to the medium was measured.

Albumin levels in the culture medium were measured using an ELISA kit (AssayPro, USA) as per the manufacturers' protocol with some modifications.

RNA was extracted from 3 pooled liver tissue model scaffolds per condition using an RNA miniprep kit (Qiagen, Manchester, UK) following the manufacturers' protocol. RNA was quantified using a Genova Plus nano spectrophotometer (Jenway, Staffordshire, UK) and 1 μg reverse transcribed using a high capacity RNA to cDNA kit (Life Technologies, Paisley, UK). PCR was performed using validated KiCqStart primers (Sigma, Poole, UK), using SYBR green (Life Technologies, Paisley, UK). PCR was performed on a QuantStudio 6 real time PCR system (Applied Biosystems, Warrington, UK). Fold change in gene expression was calculated using the ΔΔCt method selecting Beta 2 microglobulin as a house-keeping gene for all samples.

Results

Seeding Densities with Adjusted Flow Rates

The following experiment demonstrates applicability of the optimum seeding density and flow rate used for the culture of HBV in the liver tissue model.

FIG. 20 shows LDH release from cells cultured in the liver tissue model at several seeding densities. LDH increase is indicative of necrotic cell death. Adjusting the seeding densities to below the optimum of 0.6 million cells per scaffold at 1 μl/sec resulted in a large spike in LDH release at both Day 5 and 7 for 0.15 million cells at 0.2 μl/sec and 0.045 million cells at 0.2 μl/sec.

FIG. 21 shows albumin secretion from cells cultured in the liver tissue model at several seeding densities and corresponding flow rates. Albumin secretion was normalised to the number of cells seeded and resulted in comparable levels at all 3 time points.

TABLE 2
RNA quantification from pooled scaffolds at Day 7 and the 260:280
ratio from human hepatocytes cultured in the liver tissue model
at various seeding densities. Pass = Ratio above 1.7 with
sufficient quantity of RNA. This is yielded from high quality
tissue formation. Fail = Ratio below 1.7. Poor seeding of
hepatocytes results in quantities of RNA too low for qPCR.

| Cells per | RNA | | |
|---|---|---|---|
| scaffold | quantification | Ratio | |
| ×10⁶ | ug/ml | 260:280 | |
| 1.2 | 284.86 | 1.97 | Pass |
| 0.6 | 81.85 | 1.87 | Pass |
| 0.15 | 38.53 | 1.77 | Pass |
| 0.045 | 8.15 | 1.21 | Fail |

With reference to FIG. 22, qPCR data relative to the optimum conditions (0.6×10⁶ cells per scaffold at flow rate of 1 ρl/sec), demonstrated a down-regulation for all genes of interest at day 7 in culture. Note: RNA isolated from 0.045 million cells did not yield high enough quality RNA for qPCR. NTCP is essential for the entry of HBV into hepatocytes. All conditions tested showed a down-regulation of NTCP relative to the expression under from optimum conditions, indicating that the optimum conditions are important for HBV infectivity of human hepatocytes. Furthermore, down-regulation of hepatic phenotypic markers such as the genes measured indicate a hepatocyte in a de-differentiated state.

Spheroid and Single Cell Seeding With/Without Flow

An experiment was proposed to demonstrate the robustness of single cell seeding in the liver tissue model, and the need for flow during the seeding process. An optimum flow rate was used at 1 μl/sec, 0.6×10⁶ cells/scaffold.

TABLE 3
RNA quantification from pooled scaffolds at Day 7 and the 260:280
ratio from human hepatocytes seeded as spheroids or single cells
with/without flow at seeding. Pass/Fail (As above, see Table 2).

| | | | RNA | | |
|---|---|---|---|---|---|
| | Spheroids/ | Flow Rate | quantification | Ratio | |
| Cell Lot | Single cells | μl/sec | μg/ml | 260:280 | |
| Lot A | Spheroids | 1 | 6.71 | 1.10 | Fail |
| Lot B | Spheroids | 1 | 1.55 | 0.32 | Fail |
| Lot B | Single cells | 1 | 81.85 | 1.87 | Pass |
| Lot A | Spheroids | 0 | 6.22 | 1.17 | Fail |
| Lot B | Spheroids | 0 | 3.07 | 0.82 | Fail |
| Lot B | Single cells | 0 | 25.36 | 1.49 | Fail |

With reference to FIG. 23 and Table 3, spheroids did not seed into the liver tissue model, with or without flow and with reference to FIG. 25, phase contrast images show a lack of tissue formed in the channels of the scaffold.

FIG. 24 shows a fold change of several genes of interest of 0.6 million cells seeded without flow relative to optimum conditions (0.6 million cells seeded with flow). Note: RNA isolated from all spheroids formed did not yield high enough quality RNA for qPCR. Poor quality RNA is a result cif a poor culture of hepatocytes. NTCP is involved in the entry mechanism of HBV virus, Gene expression of NTCP is downregulated in all conditions in comparison with optimum conditions. Down-regulation of hepatic phenotypic markers such as the genes measured indicated a hepatocyte in a de-differentiated state.

### CONCLUSION

To conclude, 0.6 million cells at 1 μl/sec is optimum for functional tissue formation in the liver tissue model and HBV infection of primary human hepatocytes. This is scalable for 2000 cells per channel at a flow rate of 0.2 μl/min/channel, hence can be scalable to scaffolds with differing numbers of channels. With regard to spheroid and single cell seeding with/without flow, 0.6 million single cells seeded with flow out-performed cells without flow. This demonstrates that flow is necessary for cell seeding with single cells being preferable, demonstrating that the liver tissue model seeded in optimum conditions is ideal for infecting hepatocytes with HBV.

1. A method for studying an infection process in liver tissue in vitro, the method comprising:
seeding hepatocyte cells onto a scaffold in a bioreactor in order to form a liver tissue model;
delivering an infectious agent to the liver tissue model, or providing the liver tissue model pre-infected with an infectious agent; and
monitoring the infection process.

2. The method according to claim 1, wherein between about 0.4×10⁶ and about 1×10⁶ cells per scaffold are seeded at a flow rate through the bioreactor of between about 0.5 μl/s and about 2 μl/s.

3.-4. (canceled)

5. The method according to claim 1, wherein the liver tissue model produces or is capable of producing between 1000 and 100,000 TCID50/mL infectious agent per 48 hours.

6.-8. (canceled)

9. The method according to claim 1, wherein the infectious agent is delivered in a quantity of at least about 0.01 m.o.i (multiplicity of infection); or
wherein the infectious agent is delivered in a quantity of at least about 1e4 per ml.

10.-11. (canceled)

12. The method according to claim 1, further comprising seeding additional cells with the hepatocytes, wherein the additional cells are non-parenchymal liver cells.

13.-23. (canceled)

24. The method according to claim 1, wherein the infectious agent is a hepatitis virus.

25.-27. (canceled)

28. The method according to claim 1, wherein the bioreactor comprises a bioreactor well comprising the scaffold disposed therein.

29. The method according to claim 1, wherein the bioreactor comprises a fluid reservoir fluidly connected to the bioreactor well.

30. (canceled)

31. The method according to claim 1, wherein the scaffold is supported on a perfusible membrane.

32. The method according to claim 1, wherein cell culture media is flowed/perfused through the scaffold.

33. The method according to claim 1, wherein the liver tissue model is a 3-dimensional liver tissue model wherein cells are arranged in space along 3-dimensions.

34. The method according to claim 1, wherein the scaffold provides a capillary structure having microchannels.

35. (canceled)

36. The method according to claim 1, wherein the liver tissue model is capable of maintaining differentiation of the hepatocyte cells for at least 4 days.

37. The method according to claim 1, wherein the hepatocyte cells maintain NTCP and/or another viral receptor on the cell surface; and optionally where the NTCP receptor and/or another viral receptors is on a canalicular surface of the hepatocyte.

38. (canceled)

39. The method according to claim 34, wherein the hepatocytes are maintained in the physiologically correct polarity relative to the microchannels of the scaffold.

40. The method according to claim 1, wherein the hepatocytes are maintained in a physiologically relevant oxygen gradient in the scaffold.

41.-42. (canceled)

43. The method according to claim 1, comprising
priming the scaffold in the bioreactor by flowing media through the scaffold at about 37° C. for at least about 12 hours;
seeding the hepatocyte cells onto the scaffold in the bioreactor in order to form a liver tissue model, wherein the hepatocyte cells are suspended in a seeding media, and the seeding media is flowed through the scaffold at about 1 μl/s at about 37° C. for about 24 hours;
changing the media to a cell culture media at about 24 hrs and flowing the cell culture media through the scaffold at a flow rate of 1 μl/s at about 37° C. in order to maintain a cell culture of the liver tissue model;
delivering the infectious agent to the liver tissue model;
washing the hepatocyte cells at 4 hours after delivering the infectious agent by performing a media change with cell culture media; and
monitoring the infection process.

44. A screening method for identifying potential therapeutic or preventative drug candidates for the treatment or prevention of liver infection, the method comprising:
providing a liver tissue model, wherein the liver tissue model comprises hepatocytes adhered to a scaffold in a bioreactor;
delivering an infectious agent to the liver tissue model; or providing the liver tissue model pre-infected with an infectious agent;
delivering an active agent to the liver tissue model; and
monitoring the viral infection process.

45. (canceled)

46. A method of producing an infectious agent, the method comprising:
infecting a liver-tissue model with an infectious agent;
incubating the infected liver-tissue model to produce progeny of the infectious agent; and
harvesting the progeny.

47.-49. (canceled)

50. An infectious agent produced by the method according to claim 1.

51.-53. (canceled)
